(19) United States
(12) Patent Application Publication
(10) Pub. No.: US 2003/0082678 A1
(43) Pub. Date: May 1, 2003

(54) Methods and compositions for regulating body weight in bovine species

(76) Inventors: Hansen Maxwell Hsiung, Indianapolis, IN (US); Dennis Paul Smith, Indianapolis, IN (US); Xing-Yue Zhang, Indianapolis, IN (US)

Correspondence Address: ELI LILLY AND COMAPANY, Lilly Corporate Center, Patent Division/CEC, Indianapolis, IN, 46285, US

(21) Appl. No.: 09/910,180

(22) Filed: Jul. 20, 2001

(51) Int. Cl. C07K 14/705; C12P 21/02; C12N 5/06; C07H 21/04

(52) U.S. Cl. 435/69.1; 435/320.1; 435/325; 530/350; 536/23.5

(57) ABSTRACT

The present invention provides the bovine melanocortin 4 receptor protein (MC4-R), peptide and polypeptide fragments thereof, as well as nucleic acids encoding these molecules. Also provided are recombinant vectors comprising these nucleic acids, and recombinant host cells containing such vectors. Also provided are methods for identifying selective agonists and antagonists of bovine MC4-R, compounds that modulate the activity or expression of the bovine MC4-R, and methods for modifying body weight in bovine animals utilizing the bovine MC4-R as the target for intervention.

### BACKGROUND OF THE INVENTION

1. Field of the Invention

The present invention relates to the field of agriculture. More specifically, the present invention relates to melanocortin 4 receptor (MC4-R) proteins, polypeptides, and peptides of bovine species; nucleic acid molecules, e.g., RNAs and DNAs, encoding bovine melanocortin 4 receptor proteins, polypeptides, and peptides; recombinant expression vectors comprising nucleic acid molecules encoding bovine MC4-R proteins, polypeptides, and peptides; recombinant host cells containing such recombinant vectors; methods of identifying selective agonists and antagonists of bovine MC4-Rs; the agonists and antagonists identified thereby; and methods of controlling or modifying body weight in bovine species.

2. Description of Related Art

Melanocortin peptides, e.g., α-, β-, and γ-melanocyte stimulating hormones (MSH), formed by post-translational processing of proopiomelanocortin (POMC), are known to variously bind to and activate five different subtypes of melanocortin receptors, and have a broad array of physiological actions. Aside from their well known effects on adrenalcortical function (e.g., by ACTH, adrenocorticotropic hormone), and on melanocytes (e.g., by α-MSH), melanocortins have been shown to affect behavior, learning, and memory, control of the cardiovascular system, analgesia, thermoregulation, and the release of other neurohumoral agents, including prolactin, luteinizing hormone, and biogenic amines. Peripherally, melanocortins have been identified to have immunomodulatory and neurotrophic properties, and to be involved in events surrounding parturition.

The melanocortins mediate their effects through melanocortin receptors (MC-Rs), a subfamily of G-protein coupled receptors. Other than the MC1-R, which was identified as specific for α-MSH, and MC2-R, which was identified as specific for ACTH, the melanocortin receptors cloned and identified to date (MC3-R, MC4-R, and MC5-R) are thought of as “orphan” receptors, i.e., the identity of the native ligand for each receptor remains unidentified, and the physiologic function of each receptor type remains unknown.

The agouti protein is a gene product expressed in mice that is known to be involved in determining coat color, but which is also thought to play a role in obesity when its normal expression pattern is de-regulated and the protein is ubiquitously expressed. The receptor for agouti has not been identified or cloned; however, it has been observed that agouti antagonizes the MSH-induced activation of two melanocortin receptors.

Melanocortin Receptors

The first two melanocortin receptors cloned were the melanocyte MSH receptor, MC1-R, and the adrenocortical ACTH receptor, MC2-R (Mountjoy et al., 1992, Science 257: 1248-1251; Chhajlani & Wikberg, 1992, FEBS Lett. 309: 417-420). Subsequently, three additional melanocortin receptor genes were cloned which recognize the core heptapeptide sequence (MEHFRWG) of melanocortins. Two of these receptors have been shown to be expressed primarily in the brain: MC3-R (Roselli-Rehfuss et al., 1993, Proc. Natl. Acad. Sci. USA 90: 8856-8860; Gantz et al., 1993, J. Biol. Chem. 268: 8246-8250) and MC4-R (Gantz et al., 1993, J. Biol. Chem. 268: 15174-15179; Mountjoy et al., 1994, Mol. Endo. 8: 1298-1308). A fifth melanocortin receptor (originally called MC2-R) is expressed in numerous peripheral organs as well as the brain (Chhajlani et al., 1993, Biochem. Biophys. Res. Commun. 195: 866-873; Gantz et al., 1994, Biochem. Biophys. Res. Commun. 200: 1214-1220). The native ligands and functions of these latter three receptors remain unknown.

Because of their “orphan” status as receptors without an identified ligand, and the absence of any known physiological role for these new receptors, investigators have attempted to characterize the receptors in vitro by their ability to bind and respond (e.g., transduce signal) to a variety of known melanocortins (e.g., see Roselli-Rehfuss, 1993, supra; and Gantz, 1993, supra) or agonists and antagonists derived from MSH and ACTH amino acid sequences (e.g., see Hruby et al., 1995, J. Med. Chem. 38: 3454-3461; and Adan et al., 1994, Eur. J. Pharmacol. 269: 331-337). In another approach, the members of the melanocortin receptor family were differentiated on the basis of their pattern of tissue distribution as a means for hypothesizing a function (e.g., See Gantz, 1993, supra; and Mountjoy 1994, supra). For example, expression of MC1-R is localized to melanocytes and MC2-R is localized to adrenal cortical cells, whereas the MC3-R and MC4-R are found primarily in the brain but not in the adrenal cortex or melanocytes; MC4-R is not expressed in the placenta, a tissue that expresses large amounts of MC3-R. Based upon its expression pattern in the hippocampal region of the brain, a role for the MC4-R in learning and memory was proposed (Gantz, 1993, supra) but was noted to be a “pharmacological paradox” in that the MC4-R does not respond well to compounds known to have an effect on retention of learned behaviors. (Mountjoy, 1994, supra). Mountjoy 1994 further suggested that the MC4-R may participate in modulating the flow of visual and sensory information, or coordinate aspects of somatomotor control, and/or may participate in the modulation of autonomic outflow to the heart.

Thus, despite such efforts, the native ligands and function(s) of MC3-R, MC4-R and MC5-R remain elusive.

U.S. Pat. No. 5,532,347 (issued Jul. 2, 1996) to Cone and Mountjoy discloses human and mouse DNA molecules that encode MC1-R (also known in the art as α-MSH-R). The expressed human protein contains 317 amino acids.

U.S. Pat. No. 5,280,112 (issued Jan. 18, 1994) and U.S. Pat. No. 5,554,729 (issued Sep. 10, 1996), both to Cone and Mountjoy, disclose human and mouse DNA molecules that encode MC2-R (also known in the art as ACTH-R). The human MC2-R protein contains 297 amino acids.

Mountjoy, et al. (1992, Science 257:1248-1251) describe DNA molecules and the concomitant protein for human MC1-R and human MC2-R.

Chhajlani, et al. (1992, FEBS Letters 309: 417-420) also disclose a human DNA molecule comprising an open reading frame that encodes human MC1-R.

Roselli-Rehfuss, et al. (1993, Proc. Natl. Acad. Sci. USA 90: 8856-8860) disclose a cDNA clone encoding rat MC3-R cDNA.

U.S. Pat. No. 5,622,860 (issued Apr. 22, 1997) and U.S. Pat. No. 5,703,220 (issued Dec. 30, 1997) to Yamada and Gantz, disclose DNA molecules that encode human MC3-R and human MC4-R, respectively (see also Gantz, et al., 1993, J. Biol. Chem. 268(11):8246-8250).

U.S. Pat. No. 5,908,609 (issued Jun. 1, 1999) to Lee et al. discloses a human DNA molecule that encodes human MC4-R, and provides evidence that MC4-R knock-out mice gain weight, demonstrating the role and function of MC4-R in body weight regulation, and indicating that agonists or antagonists of MC4-R can be useful for treating weight disorders, or modifying body weight. The human MC4-R protein contains 332 amino acids.

PCT International Publication Wo 00/27863 (published May 18, 2000) to Macneil, et al. discloses rhesus monkey DNA molecules encoding MC4-R. The expressed rhesus monkey protein contains 332 amino acids.

A DNA molecule encoding human MC5-R was also disclosed by Mountjoy, et al. (1994, Mol. Endocrin. 8:1298-1308).

Chhajlani, et al. (1993, Biochem. Biophys. Res. Comm., 195(2): 866-873) disclose a DNA molecule which the authors state encodes MC5-R. This clone was initially designated MC2.

Fathi, et al. (1995, Neurochemical Research 20(1):107-113) also disclose a DNA molecule thought to encode human MC5-R. There are several sequence discrepancies when compared to the DNA molecule disclosed by Chhajlani, et al., supra.

Griffon, et al. (1994, Biochem. Biophys. Res. Comm., 200(2): 1007-1014) disclose DNA clones from human and rat that encode MC5-R. The human DNA sequence agrees with the human DNA sequence disclosed in Fathi et al., supra.

Gantz, et al. (1994, Biochem. Biophys. Res. Comm., 200(3):11214-11220; see also U.S. Pat. No. 5,710,265, issued Jan. 20, 1998 to Yamada and Gantz) and Labbe, et al. (1994, Biochemistry 33: 4543-4549) disclose DNA clones from mouse that encode MC5-R.

Barrett, et al. (1994, J. Mol. Endocrin. 12: 203-213) disclose DNA clones from sheep that encode MC-5R.

In rodents, MC4-R has been implicated as a key regulator of feeding behavior which regulates body weight through studies with peptide agonists and antagonists (Fan et al., 1997, Nature 385: 165-168), and with an MC4-R knock-out mouse (Huszar et al., 1997, Cell 88: 131-141). Compounds that bind to such receptors were previously identified by binding to human and/or rodent receptors and evaluated for their efficacy in rodents. However, the neuroendocrine process can differ between rodents and man. It is also expected that some compounds exhibit different binding affinities for different species homologues of the same receptor (Fong et al., 1992, J. Biol. Chem. 267:25666-25671; Hartig et al., 1992, TIPS 13:152-159).

Before a compound can be selected as a drug candidate, it must first be evaluated for a physiological effect in the target animal. It is often the case that one compound may be effective in one animal species but not in another. Previously, it has been impossible to determine if the failure was due to an altered melanocortin pathway in different species, or due to a compound having a lower affinity for the cognate receptor in one particular species.

There is a need in the art to identify and/or develop agonists and antagonists of bovine MC4 receptors, or molecules that affect expression of such receptors, for use in modifying weight gain in such animals. Bovine MC4-R nucleic acids, proteins, polypeptides, and peptides can be used to develop screens for selective MC4-R agonists and antagonists. Bovine MC4-R agonists can be useful in treating obesity in animals, if desired; bovine MC4-R antagonists can be useful in increasing weight and meat production in such animals. Such antagonists are therefore of significant commercial importance.

### SUMMARY OF THE INVENTION

The present invention addresses and meets the foregoing needs by disclosing an isolated nucleic acid fragment comprising a nucleotide sequence that codes on expression for bovine MC4-R, recombinant vectors comprising this and related nucleic acid fragments, recombinant host cells that express bovine MC-4R and/or a biologically active equivalent, and pharmacological properties of this bovine MC4-R protein.

The bovine MC4-R cDNA and protein disclosed herein can be used to develop screens for selective MC4-R agonists and antagonists, or to develop MC4-R antibodies for diagnostic kits. The bovine MC4-R antagonists may be useful to increase weight gain in farming animals, or in treating cachexia in humans (assuming that bovine MC4-R antagonists are also antagonists to human MC4-R). Bovine MC4-R agonists can be useful in treating obesity in humans or animals.

The present invention provides drug screening assays to identify compounds for the modification of body weight in bovine animal species by using bovine MC4-R as a target. The present invention also relates to compounds that modulate bovine body weight via the MC4-R. The present invention also relates to the treatment of bovine body weight disorders by targeting the MC4-R.

The invention is based, in part, on the role of MC4-R in body weight regulation. Bovid animals to which the invention disclosed herein is applicable include those within the family Bovidae, such as, for example, animals within the genus Bos, for example beef and dairy cattle (Bos taurus and Bos indicus); the genus Bison, for example the familiar bison, Bison bison; the genus Bibos, for example the guar, Bibos gaurus; and the genus Bubalus, for example the water buffalo, Bubalus bubalis.

The assays disclosed herein are designed to identify compounds or compositions that modulate MC4-R activity, i.e., compounds or compositions that act as agonists or antagonists of MC4-R, and thereby modulate weight control. Administration of agonists of MC4-R leads to weight loss; administration of antagonists leads to weight gain. Cell-based assays or non-cell based assays can be used to identify compounds that interact with, for example, specifically bind to, a bovine MC4-R extracellular domain (“ECD”). The cell-based assays have the advantage that they can be used to identify compounds that affect MC4-R biological activity (i.e., signal transduction), including the identification of compounds that do not interact with a bovine MC4-R ECD, but which act on an intracellular component of the signal transduction pathway mediated by MC4-R.

The present invention also relates to assays designed to screen for compounds or compositions that modulate bovine MC4-R gene expression. For example, cell-based assays, or cell-lysate assays (e.g., in vitro transcription or translation assays) can be used to screen for compounds or compositions that modulate MC4-R transcription (e.g., compounds that modulate expression, production, or activity of transcription factors involved in MC4-R gene expression; polynucleotides that form triple helical structures with an MC4-R regulatory region and inhibit transcription of the MC4-R gene, etc.). Alternatively, cell-based assays or cell-lysate assays can be used to screen for compounds or compositions that modulate translation of MC4-R transcripts (e.g., antisense and ribozyme molecules).

In yet another embodiment, the present invention provides cell-based assays or cell-lysate assays that can be used to test polynucleotide constructs designed to modify the expression of the bovine MC4-R gene in vivo. Such constructs include polynucleotide constructs designed for gene therapy; e.g., expression constructs or gene replacement constructs that place the bovine MC4-R gene under the control of a strong promoter system, an inducible promoter system, or a constitutive promoter system.

The present invention also encompasses agonists and antagonists of bovine MC4-R, including small molecules, large molecules, and antibodies, as well as nucleotide sequences that can be used to inhibit MC4-R gene expression (e.g., antisense and ribozyme molecules), and gene or regulatory sequence replacement constructs designed to enhance bovine MC4-R gene expression (e.g., expression constructs that place the MC4-R gene under the control of a strong promoter system). Such compounds may be used to treat body weight disorders or modify body weight in bovine animals, as desired.

The present invention also encompasses the use of such compounds and compositions, including gene therapy approaches, that modulate bovine MC4-R activity or MC4-R gene expression, to treat body weight disorders or modify body weight in bovine animals, as desired.

More specifically, in a first aspect, the present invention provides an isolated bovine melanocortin 4 receptor protein comprising the amino acid sequence shown in SEQ ID NO:2. The present invention also provides an isolated nucleic acid fragment comprising a nucleotide sequence encoding this protein.

In another aspect, the present invention provides an isolated nucleic acid fragment comprising a nucleotide sequence selected from the group consisting of:

(a) nucleotides 283 to 1281 of SEQ ID NO:1;

(b) a nucleotide sequence encoding the same protein as nucleotides 283 to 1281 of SEQ ID NO:1, but which is degenerate in accordance with the degeneracy of the genetic code; and

(c) a nucleotide sequence complementary to (a) or (b).

In another aspect, the present invention provides an isolated nucleic acid fragment that:

(a) hybridizes to the complement of nucleotides 283 to 1281 of SEQ ID NO:1 at a temperature in the range of from about 37° C. to about 42° C., a formamide concentration of about 30%, and 5× SSPE, and washing at about 42° C. in 1×-2× SSPE; and

(b) encodes a molecule that exhibits bovine melanocortin 4 receptor protein activity,

with the proviso that said isolated nucleic acid fragment is of bovine origin and comprises a previously unknown nucleotide sequence.

In another aspect, the present invention provides an isolated nucleic acid fragment that:

(a) hybridizes to the complement of nucleotides 283 to 1281 of SEQ ID NO:1 at a temperature in the range of from about 50° C. to about 65° C., a formamide concentration of about 50%, and 5× SSPE, and washing at about 50° C. to about 65° C. in 0.5× SSPE; and

(b) encodes a molecule that exhibits bovine melanocortin 4 receptor protein activity, with the proviso that said isolated nucleic acid fragment is of bovine origin and comprises a previously unknown nucleotide sequence.

In another aspect, the present invention provides a vector comprising any one of the foregoing isolated nucleic acid fragments. Such isolated nucleic acid fragments can be operably linked to regulatory elements required for expression thereof. In another aspect, the present invention provides host cells containing said vectors.

In another aspect, the present invention provides a method for expressing a bovine melanocortin 4 receptor protein in a host cell, comprising:

(a) transfecting or transforming any of the foregoing expression vectors into a suitable host cell; and

(b) culturing said host cell of step (a) under conditions that permit expression of said bovine melanocortin 4 receptor protein in said host cell.

This method can further comprise recovering said bovine melanocortin 4 receptor protein. The bovine melanocortin 4 receptor protein can comprise the amino acid sequence shown in SEQ ID NO:2.

In another aspect, the present invention provides a method for identifying a substance that binds to a bovine melanocortin 4 receptor protein, comprising:

(a) providing test cells into which has been introduced an expression cassette that directs the expression of said bovine melanocortin 4 receptor protein in said test cells, wherein said bovine melanocortin 4 receptor protein is expressed and is functional;

(b) contacting said test cells with said substance;

(c) contacting otherwise identical control cells, into which said expression cassette has not been introduced, with said substance;

(d) determining the amount of said substance bound to said test cells and said control cells; and

(e) comparing the amount of said substance bound to said test cells with the amount of said substance bound to said control cells,

wherein an increase in the amount of said substance bound to said test cells compared to the amount of said substance bound to said control cells indicates that said substance binds to said bovine melanocortin 4 receptor protein.

In yet another aspect, the present invention provides a method for identifying a substance that binds to a bovine melancortin 4 receptor protein, comprising:

(a) providing test cells into which has been introduced an expression cassette that directs the expression of said bovine melancortin 4 receptor protein in said test cells, wherein said bovine melancortin 4 receptor protein is expressed and is functional;

(b) exposing said test cells to said substance;

(c) determining the amount of cyclic AMP produced within said test cells;

(d) determining the amount of cyclic AMP produced in otherwise identical control cells into which said expression cassette has not been introduced; and

(e) comparing the amount of cyclic AMP produced in said test cells of step (c) with the amount of cyclic AMP produced in said cells of step (d), wherein an increase in the amount of cyclic AMP produced in said test cells of step (c) compared to the amount of cyclic AMP produced in said cells of step (d) indicates that said substance binds to said bovine melancortin 4 receptor protein.

In another aspect, the present invention provides a method for determining whether a substance is a potential agonist or antagonist of a bovine melanocortin 4 receptor protein, comprising:

(a) providing test cells into which has been introduced an expression cassette that directs the expression of said bovine melanocortin 4 receptor protein in said test cells, wherein said bovine melanocortin 4 receptor protein is expressed and is functional;

(b) allowing said test cells to grow for a time sufficient to permit said bovine melanocortin 4 receptor protein to be expressed;

(c) exposing said test cells to a labeled ligand of said bovine melanocortin 4 receptor protein in the presence and in the absence of said substance; and

(d) measuring the binding of said labeled ligand to said bovine melanocortin 4 receptor protein of said test cells,

wherein if the amount of binding of said labeled ligand to said bovine melanocortin 4 receptor protein of said test cells is lower in the presence of said substance than in the absence of said substance, then said substance is a potential agonist or antagonist of said bovine melanocortin 4 receptor protein.

In another aspect, the present invention provides a method for determining whether a substance binds to a bovine melanocortin 4 receptor protein, comprising:

(a) providing test cells into which has been introduced an expression cassette that directs the expression of said bovine melanocortin 4 receptor protein in said test cells, wherein said bovine melanocortin 4 receptor protein is expressed and is functional;

(b) preparing membranes containing said bovine melanocortin 4 receptor protein from said test cells and exposing said membranes to a ligand of said bovine melanocortin 4 receptor protein under conditions such that said ligand binds to said bovine melanocortin 4 receptor protein in said membranes;

(c) concurrently with, or subsequently to, step (b), contacting said membranes and said substance;

(d) measuring the amount of said ligand bound to said bovine melanocortin 4 receptor protein in said membranes in the presence and absence of said substance; and

(e) comparing the amount of said ligand bound to said bovine melanocortin 4 receptor protein in said membranes in the presence and absence of said substance,

wherein a decrease in the amount of binding of said ligand to said bovine melanocortin 4 receptor protein in said membranes in the presence of said substance indicates that said substance binds to said bovine melanocortin 4 receptor protein.

In this method, the bovine melanocortin 4 receptor protein can comprise the amino acid sequence shown in SEQ ID NO:2, and the membranes can comprise the plasma membranes of said test cells.

In a further aspect, the present invention provides a method for identifying a substance that binds to a bovine melanocortin 4 receptor protein, comprising:

(a) providing test cells into which has been introduced an expression cassette that directs the expression of said bovine melanocortin 4 receptor protein in said test cells, wherein said bovine melanocortin 4 receptor protein is expressed and is functional;

(b) preparing membranes containing said bovine melanocortin 4 receptor protein from said test cells and exposing said membranes to said substance;

(c) measuring the amount of binding of said substance to said bovine melanocortin 4 receptor protein in said membranes;

(d) comparing the amount of binding of said substance to said bovine melanocortin 4 receptor protein in said membranes from said test cells with the amount of binding of said substance to membranes from otherwise identical control cells into which said expression cassette has not been introduced,

wherein if the amount of binding of said substance to said membranes from said test cells is greater than the amount of binding of said substance to membranes from said control cells, then said substance is identified as one that binds to a bovine melanocortin 4 receptor protein.

In this method, the bovine melanocortin 4 receptor protein can comprise the amino acid sequence shown in SEQ ID NO:2, and the membranes can comprise the plasma membranes of said test cells.

In another aspect, the present invention provides a method of identifying an agonist of a bovine melanocortin 4 receptor protein, comprising:

(a) introducing into cells a first expression cassette that directs the expression of a bovine melanocortin 4 receptor protein, and a second expression cassette that directs the expression of a promiscuous G-protein, wherein both said bovine melanocortin 4 receptor protein and said promiscuous G-protein are expressed and are functional;

(b) contacting said cells and a substance suspected of being an agonist of said bovine melanocortin 4 receptor protein; and

(c) determining the amount of inositol phosphates in said cells,

wherein an increase in the amount of inositol phosphates in said cells contacted with said substance as compared to the level of inositol phosphates in said cells in the absence of said substance indicates that said substance is an agonist of said bovine melanocortin 4 receptor protein.

In this method, the bovine melanocortin 4 receptor protein can comprise the amino acid sequence shown in SEQ ID NO:2, and said promiscuous G-protein can be G₁₅ or G₁₆.

In yet another aspect, the present invention provides a method of identifying an antagonist of a bovine melanocortin 4 receptor protein, comprising:

(a) introducing into cells a first expression cassette that directs the expression of said bovine melanocortin 4 receptor protein, and a second expression cassette that directs the expression of a promiscuous G-protein, wherein both said bovine melanocortin 4 receptor protein and said promiscuous G-protein are expressed and are functional;

(b) contacting said cells and an agonist of said bovine melanocortin 4 receptor protein;

(c) concurrently with, or subsequently to, step (b), contacting said cells and a substance suspected of being an antagonist of said bovine melanocortin 4 receptor protein; and

(d) determining the amount of inositol phosphates in said cells,

wherein a decrease in the amount of inositol phosphates in said cells contacted with said substance as compared to the amount of inositol phosphates in said cells in the absence of said substance indicates that said substance is an antagonist of said bovine melanocortin 4 receptor protein.

In this method, the bovine melanocortin 4 receptor protein can comprise the amino acid sequence shown in SEQ ID NO:2, and said promiscuous G-protein can be G₁₅ or G₁₆. Furthermore, said first and second expression cassettes of step (a) can be replaced with a single expression cassette that expresses a chimeric bovine melanocortin 4 receptor protein fused at its C-terminus to said promiscuous G-protein.

In yet a further aspect, the present invention provides a method for identifying a compound useful in inducing weight loss in bovine animals, comprising:

(a) contacting a cell that expresses a functional bovine melanocortin 4 receptor protein and a test compound; and

(b) determining whether said test compound binds to said cell,

wherein a test compound that binds to said cell is identified as a compound useful in inducing weight loss in bovine animals.

In another aspect, the present invention provides a method for identifying a compound useful in inducing weight loss in bovine animals, comprising:

(a) contacting a cell that expresses a functional bovine melanocortin 4 receptor protein and a test compound; and

(b) determining whether said test compound results in bovine melanocortin 4 receptor protein-mediated activation in said cell,

wherein a test compound that results in bovine melanocortin 4 receptor protein-mediated activation in said cell is identified as a compound useful in inducing weight loss in bovine animals.

In another aspect, the present invention provides a method for identifying a compound useful in inducing weight gain in bovine animals, comprising:

(a) contacting a cell that expresses a functional bovine melanocortin 4 receptor protein, and a test compound; and

(b) determining whether said test compound results in bovine melanocortin 4 receptor protein-mediated inactivation in said cell,

wherein a test compound that results in bovine melanocortin 4 receptor protein-mediated inactivation in said cell is identified as a compound useful in inducing weight gain in bovine animals.

In another aspect, the present invention provides a method for identifying a compound useful in inducing weight gain in a bovine animal, comprising:

(a) contacting a melanocortin peptide, for example α-MSH, in the presence and absence of a test compound, and a cell that expresses a functional bovine melanocortin 4 receptor protein, and determining whether said test compound inhibits melanocortin peptide-induced activation of said bovine melanocortin 4 receptor protein; and

(b) administering said test compound to a bovine animal, and determining whether said test compound increases the body weight of said animal,

wherein a test compound that inhibits melanocortin peptide-induced activation of said bovine melanocortin 4 receptor protein in said cell and increases the body weight of said animal is identified as a compound useful for inducing weight gain in a bovine animal.

In any one of the three foregoing methods, said bovine melanocortin 4 receptor protein-mediated activation or inactivation can be determined by measuring induction of cyclic AMP. Furthermore, said cell can further contain a reporter gene operatively associated with a cyclic AMP response element (CRE) transcription factor binding site, and induction of cyclic AMP is indicated by expression of said reporter gene. Said reporter gene can be selected from the group consisting of alkaline phosphatase, chloramphenicol acetyltransferase, luciferase, glucuronide synthetase, growth hormone, β-galactosidase, and placental alkaline phosphatase.

In another aspect, the present invention provides a method for identifying a compound useful in increasing body weight in a bovine animal, comprising:

(a) contacting a bovine melanocortin 4 receptor protein having the amino acid sequence shown in SEQ ID NO:2 and a test compound with, and determining whether said test compound interacts with said bovine melanocortin 4 receptor protein;

(b) administering said test compound to a bovine animal, and determining whether said test compound increases the body weight of said animal,

wherein a test compound that interacts with said bovine melanocortin 4 receptor protein and which is effective in increasing the body weight of said animal is identified as a compound useful in increasing the body weight of a bovine animal.

In yet a further aspect, the present invention provides a method for identifying a compound useful in increasing body weight in a bovine animal, comprising:

(a) contacting a melanocortin peptide, for example α-MSH, that binds to a melanocortin 4 receptor protein and a bovine melanocortin 4 receptor protein in the presence and absence of a test compound, and determining whether said test compound inhibits interaction of said melanocortin peptide with said bovine melanocortin 4 receptor protein; and

(b) administering said test compound to a bovine animal and determining whether said test compound increases the body weight of said animal,

wherein a test compound that inhibits interaction of said melanocortin peptide with said bovine melanocortin 4 receptor protein and which increases the body weight of said animal is identified as a compound useful in increasing body weight in a bovine animal.

In either of the two foregoing methods, the bovine melanocortin 4 receptor protein can be present in a recombinant host cell or isolated cellular membrane thereof.

In yet another aspect, the present invention provides a method for identifying a compound useful in regulating body weight in a bovine animal, comprising:

(a) contacting a test compound and a cell or cell lysate containing a reporter gene operatively associated with a melanocortin 4 receptor-responsive regulatory element; and

(b) detecting expression of said reporter gene.

In another aspect, the present invention provides a method for identifying a compound useful in regulating body weight in a bovine animal, comprising:

(a) contacting a test compound and a cell or cell lysate containing bovine melanocortin 4 receptor protein transcripts; and

(b) detecting translational inhibition of said bovine melanocortin 4 receptor protein transcripts.

In another aspect, the present invention provides an antibody that binds specifically to a bovine melanocortin 4 receptor protein having the amino acid sequence shown in SEQ ID NO:2.

Further scope of the applicability of the present invention will become apparent from the detailed description provided below. However, it should be understood that the detailed description and specific examples, while indicating preferred embodiments of the present invention, are given by way of illustration only since various changes and modifications within the spirit and scope of the invention will become apparent to those skilled in the art from this detailed description.

### BRIEF DESCRIPTION OF THE TABLES AND DRAWINGS

Table 1 shows the design of the PCR primers used to isolate bovine MC4 receptor cDNA.

Table 2 is a pileup diagram of MC4-R proteins from different mammalian species. The melanocortin 4 receptors from these different species contain 332 amino acids. The bovine MC4-R protein sequence (SEQ ID NO:2) deduced from the cDNA sequence (SEQ ID NO:1) is 93.4% identical to the human MC4-R protein sequence. Seven putative transmembrane domains of the bovine MC4-R are in bold type, and are underlined. Putative extracellular and cellular domains are italicized.

Table 3 shows the functional activity of bovine MC4-R in transiently transfected human embryonic kidney 293 cells.

FIG. 1 is a hydrophobicity plot of the bovine MC4-R protein, demonstrating that the protein is a typical G-protein-coupled receptor containing seven transmembrane domains.

### DESCRIPTION OF THE SEQUENCE LISTINGS

SEQ ID NO:1: bovine MC4-R cDNA sequence;

SEQ ID NO:2: bovine MC4-R deduced amino acid sequence;

SEQ ID NO:3: MC4-A 5′-3′ PCR primer;

SEQ ID NO:4: MC4-B 3′-5′ PCR primer;

SEQ ID NO:5: putative bovine MC4-R ECD 1 amino acid sequence;

SEQ ID NO:6: putative bovine MC4-R TMD 1 amino acid sequence;

SEQ ID NO:7: putative bovine MC4-R CD 1 amino acid sequence;

SEQ ID NO:8: putative bovine MC4-R TMD 2 amino acid sequence;

SEQ ID NO:9: putative bovine MC4-R ECD 2 amino acid sequence;

SEQ ID NO:10: putative bovine MC4-R TMD 3 amino acid sequence;

SEQ ID NO:11: putative bovine MC4-R CD 2 amino acid sequence;

SEQ ID NO:12: putative bovine MC4-R TMD 4 amino acid sequence;

SEQ ID NO:13: putative bovine MC4-R ECD 3 amino acid sequence;

SEQ ID NO:14: putative bovine MC4-R TMD 5 amino acid sequence;

SEQ ID NO:15: putative bovine MC4-R CD 3 amino acid sequence;

SEQ ID NO:16: putative bovine MC4-R TMD 6 amino acid sequence;

SEQ ID NO:17: putative bovine MC4-R ECD 4 amino acid sequence;

SEQ ID NO:18: putative bovine MC4-R TMD 7 amino acid sequence;

SEQ ID NO:19: putative bovine MC4-R CD 4 amino acid sequence;

SEQ ID NO:20: rat somatostatin CRE sequence

SEQ ID NO:21: human chorionic gonadotropin (hCG) CRE sequence;

SEQ ID NO:22: CRE-2 linker.

### DETAILED DESCRIPTION OF THE INVENTION

The following detailed description of the invention is provided to aid those skilled in the art in practicing the present invention. Even so, the following detailed description should not be construed to unduly limit the present invention as modifications and variations in the embodiments discussed herein can be made by those of ordinary skill in the art without departing from the spirit or scope of the present inventive discovery.

All publications, patents, catalogs, vendor instructions, etc. cited herein are herein incorporated by reference in their entirety. These publications show the state of the art at the time of the present invention, and provide description and enablement for the present invention. Publications include any information available in any media format, including all recorded, electronic or printed formats, and include Ausubel, et al., eds., Current Protocols in Molecular Biology, John Wiley & Sons, N.Y., N.Y. (1987-1998); Coligan et al., eds., Current Protocols in Protein Science, John Wiley & Sons, Inc., N.Y., N.Y. (1995-1999); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); and Coligan, et al., eds., Current Protocols in Immunology, John Wiley & Sons, N.Y.,(1992-1999).

Definitions

The following definitions are provided to aid those of ordinary skill in the art in understanding the disclosure herein. These definitions are intended to correspond to those known in the art, and are therefore not limited to the specific definitions given, but are used according to the state of the art, as demonstrated by cited and/or contemporary publications or patents.

MC4-R polynucleotides or coding sequences: means DNA or RNA sequences, including mRNA, encoding MC4-R proteins, MC4-R peptides or polypeptides, or MC4-R fusion proteins. MC4-R polynucleotide DNA sequences encompass genomic DNA (e.g., the bovine MC4-R gene), cDNA, and synthetic DNA. The polynucleotides or coding sequences disclosed or referred to herein consist of, consist essentially of, or comprise the specific sequences disclosed.

MC4-R or MC4-R molecules: means MC4-R gene products other than mRNA transcripts, including the MC4 receptor protein per se, MC4-R polypeptides, and MC4-R peptide fragments. Fusions of MC4-R, MC4-R polypeptides, or MC4-R peptide fragments to an unrelated protein are referred to herein as MC4-R fusion proteins. A functional MC4-R refers to an MC4-R peptide, polypeptide, or protein that binds melanocortin peptides in vivo or in vitro. The MC4-R or MC4-R molecules disclosed or referred to herein consist of, consist essentially of, or comprise the specific amino acid sequences disclosed.

ECD: means “extracellular domain”.

TMD: means “transmembrane domain”.

CD: means “cytoplasmic domains”.

Active or activity: refers to form(s) of bovine MC4-R that retain, in whole or in part, the biologic and/or immunologic activities of native or naturally-occurring bovine MC4-R molecules. Elaborating further, “biological” activity refers to a biological function (either inhibitory or stimulatory) caused by a native or naturally-occurring bovine MC4-R other than the ability to induce the production of an antibody against an antigenic epitope possessed by a native or naturally-occurring bovine MC4-R. An “immunological” activity refers only to the ability to induce the production of an antibody against an antigenic epitope possessed by a native or naturally-occurring bovine MC4-R molecule. MC4-R biological activity includes, but is not limited to, ligand binding, for example α-MSH binding, and MC4-R-mediated intracellular signal transduction.

Agonist: any molecule that partially or fully stimulates, enhances, or mimics a biological activity of MC4-R. Such molecule can be natural or synthetic, endogenous or non-endogenous, and can act directly or indirectly on the MC4-R receptor to produce its effect.

Antagonist: any molecule that partially or fully blocks, inhibits, or neutralizes a biological activity of MC4-R. Such molecule can be natural, such as a melanocortin 4 receptor extracellular domain peptide that interferes with binding of a ligand to MC4-R, or synthetic, endogenous or non-endogenous, and can act directly or indirectly on the MC4-R receptor to produce its effect.

Further discussion of agonists and antagonists can be found in E. M. Ross (1996) in Hardman et al., eds., Goodman & Gilman's The Pharmacological Basis of Therapeutics, Ninth Edition, McGraw-Hill, New York, Chapter 2, pp. 29-41, incorporated by reference herein in its entirety, and to which the reader is referred.

Suitable agonist or antagonist molecules include agonist or antagonist antibodies or antibody fragments, fragments or amino acid sequence variants of native MC4-R, peptides, organic molecules, etc. Methods for identifying agonists or antagonists of MC4-R include contacting MC4-R with a candidate agonist or antagonist molecule, and measuring a detectable change in one or more biological activities normally associated with MC4-R.

Conservative substitution or conservative amino acid substitution: refers to replacement of one or more amino acid residue(s) in a peptide, polypeptide, or protein as stipulated below.

Functional fragment or functionally equivalent fragment: refers to a region, or fragment of a full length protein, or sequence of amino acids that, for example, comprises an active site, or any other conserved motif, relating to biological function of a bovine MC4-R. Functional fragments are capable of providing a biological activity substantially similar to that of a full-length MC4-R protein, or region or domain thereof, disclosed herein. Functional fragments can be produced by cloning technology, or as the natural products of alternative splicing mechanisms.

Fusion protein: denotes a hybrid peptide, polypeptide, or protein molecule not found in nature, comprising a translational fusion or enzymatic fusion in which two or more different peptides, polypeptides, or proteins, or fragments thereof, are covalently linked in a single polypeptide chain.

Homolog or homologous: describes the relationship between different nucleic acid molecules or amino acid sequences such that said molecules or sequences are related by partial identity or similarity at one or more regions within said molecules or sequences.

Host cell: refers to any eucaryotic, procaryotic, fusion or other cell or pseudo cell, or membrane-containing construct, that is suitable for propagating and/or expressing an isolated nucleic acid that is introduced into such host cell by any suitable means known in the art (for example, but not limited to, transformation or transfection, or the like), or which is induced to express an endogenous nucleic acid encoding an MC4-R peptide, polypeptide, protein, or fusion protein, etc., according to the present invention. The cell can be part of a tissue or organism, isolated in culture, immobilized, or in any other suitable form.

Hybridization: refers to the process by which a partially or completely single-stranded nucleic acid molecule joins with a complementary strand through nucleotide base pairing. Hybridization can occur under conditions of low, moderate or high stringency. The degree of hybridization depends upon, for example, the degree of homology, the stringency conditions, and the length of hybridizing strands, as is known in the art. Nucleic acids encompassed by the present invention can hybridize fully, i.e., along 100% of their own length, or only partially along their own length. Furthermore, such hybridizing nucleic acids can hybridize fully, i.e., along 100% of the length of nucleotide sequences disclosed herein, along 100% of the length of the complements of the nucleic acid sequences disclosed herein, or only partially along these lengths of nucleotide sequences.

Isolated nucleic acid fragment or molecule: a nucleic acid fragment or molecule, for example DNA, RNA, or both, that has been removed from its native or naturally occurring environment. Such nucleic acid fragments do not include whole chromosomes, or the entire chromosomal DNA of a cell. For example, recombinant nucleic acid fragments or molecules contained or generated in culture, in a vector, and/or in a host cell are considered isolated for the purposes of the present invention. Further examples of isolated nucleic acid fragments or molecules include recombinant nucleic acid molecules maintained in heterologous host cells, or purified (partially or substantially) nucleic acid molecules in solution. Isolated RNA molecules include in vivo or in vitro RNA transcripts of the DNA molecules of the present invention. Isolated nucleic acid molecules according to the present invention further include nucleic acid molecules produced synthetically, or purified from or provided in cells containing such synthetic nucleic acids, where the nucleic acid exists in other than a naturally occurring form, quantitatively or qualitatively.

Isolated peptide, polypeptide, or protein: refers to a state of isolation such that the peptide, etc., is not in a naturally occurring form, and/or has been purified to remove at least some portion of cellular or non-cellular molecules with which it is naturally associated. “Isolated” may include the addition of other functional or structural peptides, etc., for a specific purpose.

The term “isolated” used in reference to at least one antibody of the invention describes an antibody that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver staining. “Isolated antibody” includes the antibody in situ within recombinant cells since at least one component of the antibody's natural environment will not be present. Ordinarily, however. isolated antibody will be prepared by at least one purification step.

The term “stringency” refers to hybridization conditions for nucleic acids in solution. High stringency conditions disfavor non-homologous base pairing. Low stringency conditions have much less of this effect. Stringency may be altered, for example, by changes in temperature and/or salt concentration, or other conditions, as is well known in the art.

Typical “high stringency” conditions comprise, for example, hybridizing at a temperature in the range of from about 50° C. to about 65° C., a formamide concentration of about 50%, and 5× SSPE, and washing at about 50° C. to about 65° C. in 0.5× SSPE. Typical “low stringency” conditions comprise, for example, hybridizing a temperature in the range of from about 37° C. to about 42° C., a formamide concentration of about 30%, and 5× SSPE, and washing at about 42° C. in 1×-2× SSPE. “SSPE” comprises a hybridization and wash solution. A 1× SSPE solution contains 180 mM NaCl, 9 mM Na2HPO4, 0.9 mM NaH2PO4 and 1 mM EDTA, pH 7.4.

Variant: with reference to the bovine MC4-R protein having the amino acid sequence shown in SEQ ID NO:2, or a peptide or polypeptide fragment thereof, “variant” means an MC4-R peptide, polypeptide, or protein, respectively, having at least about 80% amino acid sequence identity to said amino acid sequence, and exhibiting at least one of the activities associated with said MC4-R peptide, polypeptide, or protein sequence, respectively.

The term “variant” in reference to an MC4-R polynucleotide means an active MC4-R-encoding nucleic acid molecule as defined below, having at least about 65% nucleic acid sequence identity with the MC4-R-encoding nucleotide sequence shown in SEQ ID NO: 1, or corresponding peptide- or polypeptide-encoding fragment thereof. Variants specifically exclude or do not encompass the native bovine MC4-R-encoding nucleotide sequence shown in SEQ ID NO:1, or peptide- or polypeptide-encoding fragments thereof, as well as those prior art sequences that share 100% sequence identity with these nucleotide sequences.

Percent (%) nucleic acid sequence identity: with respect to the MC4-R nucleic acid sequences identified herein, percent (%) nucleic acid sequence identity is defined as the percentage of nucleotides in a candidate sequence that is identical with the nucleotides in the corresponding MC4-R sequence after aligning the sequences and introducing gaps, if necessary, to achieve the maximum percent sequence identity. Alignment for purposes of determining percent nucleic acid sequence identity can be achieved in various ways that are within the skill in the art. For example, percent nucleic acid sequence identity can be determined using publicly available computer software such as ALIGN, Align-2, Megalign (DNASTAR), or BLAST (e.g., Blast, Blast-2) software. Those skilled in the art can determine appropriate parameters for measuring alignment, including any algorithms needed to achieve maximal alignment over the full length of the sequences being compared. For purposes herein, however, percent nucleic acid identity values are generated using the WU-BLAST-2 (BlastN module) computer program (Altschul et al., 1996, Methods in Enzymology: 460-480). Most of the WU-BLAST-2 search parameters are set to the default values. Those not set default values, i.e., the adjustable parameters, are set with the following values: overlap span=1, overlap fraction=0.125, word threshold (T)=11, and scoring matrix=BLOSUM62. For purposes herein, a percent nucleic acid sequence identity value is determined by dividing (a) the number of matching identical nucleotides between the nucleic acid sequence of the MC4-R-encoding nucleic acid molecule of interest and the comparison nucleic acid molecule of interest (i.e., the sequence against which the MC4-R-encoding nucleic acid molecule of interest is being compared) as determined by WU-BLAST-2 by (b) the total number of nucleotides of the MC4-R-encoding nucleic acid molecule of interest.

Percent (%) amino acid sequence identity: with respect to the MC4-R amino acid sequences identified herein, percent (%) amino acid sequence identity is defined as the percentage of amino acid residues in a candidate sequence that is identical with the amino acid residues in a corresponding MC-R peptide, polypeptide, or protein sequence, after aligning the sequences and introducing gaps, if necessary, to achieve the maximum percent sequence identity, and not considering any conservative amino acid substitutions as part of the sequence identity. Alignment for purposes of determining percent amino acid sequence identity can be achieved in various ways that are within the skill in the art. For instance, one can use publicly available computer software such as ALIGN, ALIGN-2, Megalign (DNASTAR) or BLAST (e.g., Blast, Blast-2, WU-Blast-2) software. Those skilled in the art can determine appropriate parameters for measuring alignment, including any algorithms needed to achieve maximal alignment over the full length of the sequences being compared. For example, the percent identity values can be generated using WU-BLAST-2 (Altschul et al., 1996, Methods in Enzymology 266: 460-480). Most of the WU-BLAST-2 search parameters are set to the default values. Those not set to default values, i.e., the adjustable parameters, are set with the following values: overlap span=1. overlap fraction=0.125: word threshold (T)=11, and scoring matrix=BLOSUM 62. For purposes herein, a percent amino acid sequence identity value is determined by dividing the number of matching identical amino acid residues between the amino acid sequence of the MC4-R peptide, polypeptide, or protein of interest and the comparison amino acid sequence of interest (i.e., the sequence against which the MC4-R sequence of interest is being compared) as determined by WU-BLAST-2 by (b) the total number of amino acid residues of the MC4-R sequence of interest, respectively.

Nucleic Acid Molecules

Using the information provided herein, such as the nucleotide sequences encoding at least 80-100% of the contiguous amino acids of SEQ ID NO:2, specified fragments or variants thereof, or a vector comprising at least one of these sequences, a nucleic acid molecule of the present invention encoding an MC4-R peptide, polypeptide, or protein can be obtained using well-known methods.

Nucleic acid molecules of the present invention can be in the form of RNA, such as mRNA, hnRNA, t-RNA, or any other form, or in the form of DNA, including, but not limited to, cDNA or genomic DNA obtained by cloning, or produced synthetically, or any combination thereof, that code on expression for an MC4-R peptide, polypeptide, or protein. The DNA can be triple-stranded, double-stranded or single-stranded, or any combination thereof. The entire or any portion of at least one strand of the DNA or RNA can be the coding strand, also known as the sense strand, or it can be the non-coding strand, also referred to as the anti-sense strand. Nucleic acids of the present invention further include those that are partially or fully complementary to these coding or non-coding strands.

Isolated nucleic acid molecules of the present invention include nucleic acid molecules comprising an open reading frame (ORF) of a genomic DNA sequence, including introns, if any; nucleic acid molecules comprising the coding sequence for an MC4-R peptide, polypeptide, or protein, such as a cDNA or synthetic DNA; and nucleic acid molecules that comprise a nucleotide sequence substantially different from those described herein, but which, due to the degeneracy of the genetic code, still encode an MC4-R peptide, polypeptide, or protein as disclosed herein. Of course, the genetic code is well known in the art. Thus, it would be routine for one skilled in the art to generate such degenerate nucleic acid variants that code for MC4-R peptides, polypeptides, or proteins of the present invention. See, e.g., Ausubel, et al., supra. Such nucleic acid variants are included in the present invention.

In one embodiment, the isolated nucleic acid comprises RNA or DNA having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% sequence identity to (a) an RNA or DNA molecule encoding an MC4-R peptide, polypeptide, or protein as disclosed herein, or (b) the complement of the RNA or DNA molecule of (a), with the proviso that each and every one of said individual RNA or DNA molecules having a sequence identity in the range from at least about 80% to at least about 99% as compared to the nucleic acid molecules disclosed herein is not one known in the art at the time of filing of this application, and further, is preferably of bovine origin.

In another aspect, the present invention encompasses isolated nucleic acid molecules encoding an MC4-R peptide, polypeptide, or protein, comprising nucleotide sequences that hybridize to the complement of SEQ ID NO:1, or the complements of the nucleic acid sequences encoding the MC4-R domains shown in SEQ ID Nos:5 to 19. Such DNA can hybridize fully, i.e., along 100% of its length, or only partially. Furthermore, such hybridizing DNA can hybridize fully, i.e., along 100% of the length of the complement of SEQ ID NO:1 or along 100% of the length of the complements of the nucleic acid sequences encoding the MC4-R domains shown in SEQ ID Nos:5 to 19, or only partially thereto. In any of these cases, such hybridizing nucleic acid molecules code on expression for molecules that exhibit bovine MC4-R peptide, polypeptide, or protein biological activity, as appropriate, such as melanocortin peptide binding, MC4-R-mediated intracellular signal transduction, etc. Preferably, hybridization occurs under stringent hybridization and wash conditions.

In another aspect, the present invention encompasses an isolated nucleic acid fragment or molecule comprising a nucleotide sequence encoding a protein having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% sequence identity to (a) the full-length protein encoded by the cDNA having the nucleotide sequence shown in SEQ ID NO:1, or (b) the complement of the nucleotide sequence of (a). In any of these cases, such coding strand nucleic acid molecules code on expression for molecules that exhibit bovine MC4-R protein biological activity, such as melanocortin peptide binding, MC4-R-mediated intracellular signal transduction, etc. In a preferred embodiment, the isolated nucleic acid molecule encodes the same full length polypeptide as encoded by the cDNA having the nucleotide sequence shown in SEQ ID NO:1.

In a further aspect, the present invention encompasses an isolated nucleic acid molecule obtainable by hybridizing a test DNA molecule under stringent conditions with (a) a DNA molecule encoding an MC4-R peptide such as any one of those having the amino acid sequences shown in SEQ ID Nos:5-19, encoding a polypeptide as disclosed herein, or encoding the MC4-R protein having the amino acid sequence shown in SEQ ID NO:2, or with (b) the complement of the DNA molecule of (a), wherein the isolated nucleic acid molecule has at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% sequence identity to (a) or (b), and isolating the test DNA molecule. In any of the cases where the test DNA molecule hybridizes to (b), i.e., where such test DNA molecule is a coding strand nucleic acid molecule, such hybridizing coding strand test DNA molecule codes on expression for a molecule that exhibits the same, or substantially the same, activity as the bovine MC4-R peptide, polypeptide, or protein encoded by the complement of (b) (i.e., the complementary coding strand (a) nucleic acid molecule), such as ligand binding, for example, melanocortin peptide binding, MC4-R-mediated intracellular signal transduction, etc.

In yet a further aspect, the present invention encompasses an isolated nucleic acid molecule comprising: (a) RNA or DNA encoding a peptide, polypeptide, or protein scoring at least about 80% positives, preferably at least about 81% positives, more preferably at least about 82% positives, yet more preferably at least about 83% positives, yet more preferably at least about 84% positives, yet more preferably at least about 85% positives, yet more preferably at least about 86% positives, yet more preferably at least about 87% positives, yet more preferably at least about 88% positives, yet more preferably at least about 89% positives, yet more preferably at least about 90% positives, yet more preferably at least about 91% positives, yet more preferably at least about 92% positives, yet more preferably at least about 93% positives, yet more preferably at least about 94% positives, yet more preferably at least about 95% positives, yet more preferably at least about 96% positives, yet more preferably at least about 97% positives, yet more preferably at least about 98% positives, yet more preferably at least about 99% positives, when compared with the corresponding amino acid sequence within residues about 1 to about 322, inclusive, of SEQ ID NO:2, or (b) the complement of the RNA or DNA of (a).

The MC4-R amino acid sequence shown in SEQ ID NO:2 was deduced from the cDNA sequence shown in SEQ ID NO:1.

Nucleic Acid Fragments

The present invention encompasses fragments of the presently disclosed MC4-R polynucleotide sequences, useful as, for example, hybridization probes, PCR primers, or for encoding peptide or polypeptide fragments of MC4-R comprising a binding site for an anti-MC4-R antibody using the methods disclosed herein, or corresponding to the ECDs, TMDs, or CDs of the MC4-R protein.

Other useful fragments of the present bovine MC4-R nucleic acid sequences include antisense or sense oligonucleotides comprising a single-stranded nucleic acid sequence (either RNA or DNA) capable of binding to target MC4-R molecule mRNA (sense) of MC4-R DNA (anti-sense) sequences. Antisense or sense oligonucleotides, according to the present invention, comprise a fragment of the coding region of MC4-R DNA. Such a fragment generally comprises at least about 12 nucleotides, preferably from about 12 to about 30 nucleotides, more preferably from about 12 to about 18 nucleotides. The ability to derive an antisense or a sense oligonucleotide, based upon a cDNA sequence encoding a given protein, is described in, for example, Stein and Cohen, 1988, Cancer Rex @2659 and van der Krol et al., 1988, BioTechniques 6: 958 (1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. The antisense oligonucleotides thus may be used to block expression of MC4-R molecules. Antisense or sense oligonucleotides further comprise oligonucleotides having modified sugar-phosphodiester backbones (or other sugar linkages, such as those described in WO 91/106629), and wherein such sugar linkages are resistant to endogenous nucleases. Such oligonucleotides with resistant sugar linkages are stable in vivo (i.e., capable of resisting enzymatic degradation), but retain sequence specificity to be able to bind to target nucleotide sequences.

Other examples of sense or antisense oligonucleotides include those oligonucleotides that are covalently linked to organic moieties, such as those described in WO 90/10048, and other moieties that increase affinity of the oligonucleotide for a target nucleic acid sequence, such as poly-L-lysine. Further still, intercalating agents such as ellipticine, and alkylating agents or metal complexes, can be attached to sense or antisense oligonucleotides to modify binding specificities of the antisense or sense oligonucleotide for the target nucleotide sequence. Antisense or sense oligonucleotides can be introduced into a cell containing the target nucleic acid sequence by any gene transfer method, including, for example, CaPO₄ mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MSV, N2 (a retrovirus derived from M-MuLV), or the double copy vectors designated CDTSA, CTSB and DCTSC (see WO 90/13641).

Sense or antisense oligonucleotides can also be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a sense or an antisense oligonucleotide can be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

By a fragment of an isolated nucleic acid molecule is meant a molecule having at least about 10 nucleotides of the nucleotide sequence shown in SEQ ID NO:1, and is intended to mean fragments at least about 10 nucleotides in length, preferably about 18 to about 22 nucleotides in length, which are useful, inter alia, as diagnostic probes and primers as described herein. By a fragment at least about 10 nucleotides in length, for example, is intended fragments that include about 10 or more contiguous nucleotides from the nucleotide sequence shown in SEQ ID NO:1. Preferred probes of the present invention comprise at least about 18, preferably about 18-22, contiguous nucleotides of SEQ ID NO:1, or the complement thereof.

The present invention also provides subsequences of full-length nucleic acids encompassed by the present invention. Any number of subsequences can be obtained by reference to SEQ ID NO:1, or the complementary sequence thereto, using primers that selectively hybridize, under stringent conditions to: at least two sites in the polynucleotides of the present invention, or to two sites within the nucleic acid that flank and comprise a polynucleotide of the present invention, or to a site within a polynucleotide of the present invention and a site within the nucleic acid that comprises it. A variety of methods for obtaining 5′ and/or 3′ ends is well known in the art. See, e.g., RACE (Rapid Amplification of Complementary Ends) as described in M. A. Frohman, 1990, PCR Protocols: A Guide to Methods and Applications, M. A. Innis, et al., Eds., Academic Press, Inc., San Diego, Calif., pp. 28-38; see also, U.S. Pat. No. 5,470,722, and Ausubel, et al., 1989-1999, Current Protocols in Molecular Biology, Chapter 15, John Wiley & Sons, N.Y. Thus, the present invention provides MC4-R polynucleotides having the sequence of the MC4-R gene, nuclear transcript, cDNA, or complementary sequences, and/or subsequences thereof. Such subsequences include, for example, those encoding ECDs, TMDs, and CDs of the present bovine MC4-R protein.

Primer sequences can be obtained by reference to a contiguous subsequence of a polynucleotide of the present invention. Primers are chosen to selectively hybridize, under PCR amplification conditions, to a polynucleotide of the present invention in an amplification mixture comprising a genomic and/or cDNA library from the same or closely related animal species. Generally, the primers are complementary to a subsequence of the amplified nucleic acid. In some embodiments, the primers will be constructed to anneal at their 5′ terminal ends to the codon encoding the carboxy or amino terminal amino acid residue (or the complements thereof) of the polynucleotides of the present invention. The primer length in nucleotides is selected from the group of integers consisting of from at least about 15 to about 50. Thus, the primers can be at least about 15, about 18, about 20, about 25, about 30, about 40, or about 50 nucleotides in length, or any range or value therein. Preferred primers are about 18 to about 22 nucleotides in length. A non-annealing sequence at the 5′ end of the primer (a “tail”) can be added, for example, to introduce a cloning site at the terminal ends of the amplified DNA.

The amplification primers can optionally be elongated in the 3′ direction with additional contiguous or complementary nucleotides from the polynucleotide sequences. The number of nucleotides by which the primers can be elongated is selected from the group of integers consisting of from at least about 1 to at least about 25. Thus, for example, the primers can be elongated with an additional about 1, about 5, about 10, or about 15 nucleotides, or any range or value therein. Those of ordinary skill in the art will recognize that a lengthened primer sequence can be employed to increase specificity of binding (i.e., annealing) to a target sequence, or to add useful sequences, such as links or restriction sites (See e.g., Ausubel, supra, Chapter 15).

The amplification products can be translated using expression systems well known to those of skill in the art, as discussed infra. The resulting translation products can be confirmed as peptides or polypeptides of the present invention by, for example, assaying for the appropriate biological activity, or verifying the presence of one or more linear epitopes specific to a peptide or polypeptide of the present invention. Methods for protein synthesis from PCR-derived templates are known in the art (See e.g., Ausubel, supra, Chapters 9, 10, 15; Coligan, Current Protocols in Protein Science, supra, Chapter 5), and kits therefor are available commercially. See, e.g., Amersham Life Sciences, Inc., Catalog '97, p. 354.

Nucleic Acid Fragments That Selectively Hybridize to Polynucleotides Described Herein

The present invention provides nucleic acid fragments that hybridize under selective hybridization conditions to polynucleotides disclosed herein, e.g., SEQ ID NO:1, and to related polynucleotides. Such nucleic acid fragments can be used to detect, isolate, and/or quantify nucleic acids comprising polynucleotides of the present invention, including polynucleotides related thereto. For example, such hybridizing nucleic acid fragments can be used to identify, isolate, or amplify partial or full-length clones in a deposited library. Such clones can be genomic DNA or RNA, or cDNA sequences, or complementary to a cDNA from a mammalian nucleic acid library.

Preferably, the cDNA library comprises at least 80% full-length sequences, preferably at least 85% or 90% full-length sequences, and more preferably at least 95% full-length sequences. The cDNA library can be normalized to increase the representation of rare sequences. Low stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity, and can be employed to identify orthologous or paralogous sequences.

Polynucleotides encompassed by the present invention include those that encode one or more domains or epitopes of the MC4-R protein disclosed herein. The polynucleotides of the present invention encompass nucleic acid sequences that can be employed for selective hybridization to a polynucleotide encoding a peptide or polypeptide of the present invention.

Complementary Polynucleotides

As indicated above, the present invention provides isolated nucleic acid fragments comprising MC4-R polynucleotides, wherein the polynucleotides are complementary to the polynucleotides described herein. As those of skill in the art will recognize, complementary sequences can base pair throughout the entirety of their length with such polynucleotides (i.e., have 100% sequence identity over their entire length), or can be partially complementary. Complementary bases associate through hydrogen bonding in double-stranded nucleic acids. For example, the following base pairs are complementary: guanine and cytosine; adenine and thymine; and adenine and uracil (See, e.g., Ausubel, supra, Chapter 67; or Sambrook, supra).

Recombinant Methods for Constructing Nucleic Acids

The isolated nucleic acid compositions of the present invention, such as RNA, cDNA, genomic DNA, synthetic DNA, or hybrids thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and the construction of cDNA and genomic libraries, are well known to those of ordinary skill in the art. (See, e.g., Ausubel, supra, Chapters 1-7; or Sambrook).

Nucleic Acid Screening and Isolation Methods

A cDNA or genomic library can be screened using a probe based upon the sequence of an MC4-R polynucleotide of the present invention, such as those disclosed herein. Probes can be used to hybridize with genomic DNA or cDNA sequences to isolate homologous genes in the same or different organisms. Those of ordinary skill in the art will appreciate that various degrees of stringency of hybridization can be employed in the assay, and either the hybridization or the wash medium can be stringent. As the conditions for hybridization become more stringent, there must be a greater degree of complementarity between the probe and the target for duplex formation to occur. Temperature, ionic strength, pH, and the presence of a partially denaturing solvent such as formamide can control the degree of stringency. Changing the polarity of the reactant solution through, for example, manipulation of the concentration of formamide within the range of 0% to 50% conveniently varies the stringency of hybridization. The degree of complementarity (sequence identity) required for detectable binding will vary in accordance with the stringency of the hybridization medium and/or wash medium. The degree of complementarity will optimally be 100%; however, it should be understood that minor sequence variations in the probes and primers may be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; 4,795,699 and 4,921,794 to Tabor, et al; 5,142,033 to Innis; 5,122,464 to Wilson, et al.; 5,091,310 to Innis; 5,066,584 to Gyllensten, et al; 4,889,818 to Gelfand, et al; 4,994,370 to Silver, et al; 4,766,067 to Biswas; 4,656,134 to Ringold) and RNA mediated amplification which uses antisense RNA to the target sequence as a template for double-stranded DNA synthesis (U.S. Pat. No. 5,130,238 to Malek, et al, with the tradename NASBA), the entire contents of which are herein incorporated by reference. (See, e.g., Ausubel, supra, Chapter 15; or Sambrook, supra)

For instance, polymerase chain reaction (PCR) technology can be used to amplify the sequences of polynucleotides of the present invention and related genes and cDNAs directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, Sambrook, and Ausubel (e.g., Chapter 15) supra, as well as Mullis, et al., U.S. Pat. No. 4,683,202 (1987), and Innis, et al., Eds., 1990, PCR Protocols, A Guide to Methods and Applications, Academic Press Inc., San Diego, Calif. Commercially available kits for genomic PCR amplification are known in the art. See, e.g., Advantage-GC Genomic PCR Kit (Clontech). The T4 gene 32 protein (Boehringer Mannheim) can be used to improve the yield of long PCR products.

Bovine MC4-R Peptide, Polypeptide, and Protein Variants

The present invention further encompasses amino acid variants of the peptide, polypeptide, and protein sequences disclosed herein.

Exemplary sequences are provided in SEQ ID NOs:1 and 2, and the sequence domains shown in Table 2, as well as their corresponding encoding nucleotide sequences. The peptides, polypeptides, and proteins of the present invention, or variants thereof, can comprise any number of contiguous amino acid residues. The subsequence of contiguous amino acids derived from peptides, polypeptides, or proteins disclosed herein can be at least about 20, at least about 30, at least about 40, at least about 50, at least about 60, at least about 70, at least about 80, or at least about 90 amino acids in length. Furthermore, the number of contiguous amino acid residues in such subsequences can be any integer selected from the group consisting of from 1 to 20, such as 2, 3, 4, or 5.

In a further aspect, the present invention encompasses an isolated MC4-R protein, and isolated MC4-R ECD, TMD, or CD peptides, comprising an amino acid sequence having at least about 80% sequence identity, preferably at least about 81% sequence identity, more preferably at least about 82% sequence identity, yet more preferably at least about 83% sequence identity, yet more preferably at least about 84% sequence identity, yet more preferably at least about 85% sequence identity, yet more preferably at least about 86% sequence identity, yet more preferably at least about 87% sequence identity, yet more preferably at least about 88% sequence identity, yet more preferably at least about 89% sequence identity, yet more preferably at least about 90% sequence identity, yet more preferably at least about 91% sequence identity, yet more preferably at least about 92% sequence identity, yet more preferably at least about 93% sequence identity, yet more preferably at least about 94% sequence identity, yet more preferably at least about 95% sequence identity, yet more preferably at least about 96% sequence identity, yet more preferably at least about 97% sequence identity, yet more preferably at least about 98% sequence identity, yet more preferably at least about 99% sequence identity, to the MC4-R protein of the present invention (SEQ ID NO:2), and ECDs, TMDs, and CDs thereof (SEQ ID NOs:5-19), and nucleic acids encoding these MC4-R molecules, with the proviso that that each and every one of said individual peptide or protein molecules having a sequence identity in the range from at least about 80% to at least about 99% as compared to the MC4-R molecules disclosed herein is not one known in the art at the time of filing of this application, and further, is preferably of bovine origin.

In a further aspect, the present invention relates to an isolated protein comprising an amino acid sequence scoring at least about 80% positives, preferably at least about 81% positives, more preferably at least about 82% positives, yet more preferably at least about 83% positive, yet more preferably at least about 84% positives, yet more preferably at least about 85% positives, yet more preferably at least about 86% positives, yet more preferably at least about 87% positives, yet more preferably at least about 88% positives, yet more preferably at least about 89% positives, yet more preferably at least about 90% positives, yet more preferably at least about 91% positives, yet more preferably at least about 92% positives, yet more preferably at least about 93% positives, yet more preferably at least about 94% positives, yet more preferably at least about 95% positives, yet more preferably at least about 96% positives, yet more preferably at least about 97% positives, yet more preferably at least about 98% positives, yet more preferably at least about 99% positives, when compared with the amino acid sequence of residues shown in SEQ ID NO:2.

The present invention encompasses biologically active variants of the MC4-R peptides, polypeptides, and proteins disclosed herein. Biological activity includes, for example, melanocortin receptor-mediated signal transduction, etc. Such biologically active peptides, polypeptides, and proteins have activity that is at least about 20%, 30%, or 40%, preferably at least about 50%, 60%, or 70%, and most preferably at least about 80%, 90%, or 95%-100% of that of the corresponding native (non-synthetic) MC4-R peptide, polypeptide, or protein. Furthermore, the ligand binding specificity of such variant MC4-R molecules is substantially similar to that of the corresponding native (non-synthetic) peptide, polypeptide, or protein. Typically, the ligand binding specificity will be at least about 30%, 40%, or 50% that of the corresponding native (non-synthetic) peptide, polypeptide, or protein, and more preferably at least about 60%, 70%, 80%, or 90%-100% thereof. Methods of assaying and quantifying measures of biological activity and ligand binding by melanocortin receptors are well known to those of skill in the art.

The term “amino acid” is used herein in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids such as norleucine, β-alanine, ornithine, etc.; and chemically synthesized compounds having properties known in the art to be characteristic of amino acids. As used herein, the term “proteogenic” indicates that the amino acid can be incorporated into a peptide, polypeptide, or protein in a cell through a metabolic pathway.

The incorporation of non-natural amino acids, including synthetic non-native amino acids, substituted amino acids, or one or more D-amino acids into the MC4-R peptides, polypeptides, or proteins of the present invention (“D- MC4-R molecules”) is advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. When it is desirable to allow the peptide, etc., to remain active for only a short period of time, the use of L-amino acids therein will permit endogenous peptidases, proteases, etc., in a cell to digest the molecule in vivo, thereby limiting the cell's exposure to the molecule. Additionally, D-peptides, etc., cannot be processed efficienty for major histocompatibility complex class II-restricted presentation to T helper cells, and are therefore less likely to induce humoral immune responses in the whole organism.

In addition to using D-amino acids, those of ordinary skill in the art are aware that modifications in the amino acid sequence of a peptide, polypeptide, or protein can result in equivalent, or possibly improved, second generation peptides, etc., that display equivalent or superior functional characteristics when compared to the original amino acid sequences. Alterations in the MC4-R peptides, polypeptides, or proteins of the present invention can include one or more amino acid insertions, deletions, substitutions, truncations, fusions, shuffling of subunit sequences, and the like, either from natural mutations or human manipulation, provided that the sequences produced by such modifications have substantially the same (or improved or reduced, as may be desirable) activity(ies) as the naturally occurring counterpart sequences disclosed herein.

One factor that can be considered in making such changes is the hydropathic index of amino acids. The importance of the hydropathic amino acid index in conferring interactive biological function on a protein has been discussed by Kyte and Doolittle (1982, J. Mol. Biol., 157: 105-132). It is accepted that the relative hydropathic character of amino acids contributes to the secondary structure of the resultant protein. This, in turn, affects the interaction of the protein with molecules such as enzymes, substrates, receptors, ligands, DNA, antibodies, antigens, etc. Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide, polypeptide, or protein can be substituted for other amino acids having a similar hydropathic index or score and produce a resultant peptide, etc., having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant peptide, etc., having similar biological activity, i.e., still retaining correct biological function. In making such changes, amino acids having hydropathic indices within ±2 are preferably substituted for one another, those within +1 are more preferred, and those within ±0.5 are most preferred.

As outlined above, amino acid substitutions in the MC4-R molecules of the present invention can be based on the relative similarity of the amino acid side-chain substituents, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences. Since small peptides, etc., can be easily produced by conventional solid phase synthetic techniques, the present invention includes peptides, etc., such as those discussed herein, containing the amino acid modifications discussed above, alone or in various combinations. To the extent that such modifications can be made while substantially retaining the activity of the peptide, etc., they are included within the scope of the present invention. The utility of such modified peptides, etc., can be determined without undue experimentation by, for example, the methods described herein.

While biologically functional equivalents of the present MC4-R molecules can have any number of conservative or non-conservative amino acid changes that do not significantly affect their activity(ies), or that increase or decrease activity as desired, 40, 30, 20, 10, 5, or 3 changes, such as 1-30 changes or any range or value therein, may be preferred. In particular, 10 or fewer amino acid changes may be preferred. More preferably, seven or fewer amino acid changes may be preferred; most preferably, five or fewer amino acid changes may be preferred. The encoding nucleotide sequences (gene, plasmid DNA, cDNA, synthetic DNA, or mRNA, for example) will thus have corresponding base substitutions, permitting them to code on expression for the biologically functional equivalent forms of the MC4-R molecules. In any case, the MC4-R peptides, polypeptides, or proteins exhibit the same or similar biological or immunological activity(ies) as that (those) of the MC4-R molecules specifically disclosed herein, or increased or reduced activity, if desired.

The activity(ies) of the variant MC4-R molecules can be determined by the methods described herein or as are known in the art. Variant MC4-R molecules biologically functionally equivalent to those specifically disclosed herein have activity(ies) differing from those of the presently disclosed molecules by about ±50% or less, preferably by about ±40% or less, more preferably by about 30% or less, more preferably by about ±20% or less, more preferably by about ±10% or less, and even more preferably by about ±5% or less, when assayed by the methods disclosed herein, or as are known in the art.

Amino acids in an MC4-R molecule of the present invention that are essential for activity can be identified by methods known in the art, such as site-directed mutagenesis or alanine-scanning mutagenesis (Cunningham and Wells, 1989, Science 244:1081-1085). The latter procedure introduces single alanine mutations at every residue in the molecule. The resulting mutant molecules are then tested for biological activity. Sites that are critical for ligand binding can also be identified by structural analysis such as crystallization, nuclear magnetic resonance, or photoaffinity labeling (Smith, et al., 1992, J. Mol. Biol. 224:899-904 (1992) and de Vos, et al., Science 255:306-312 (1992)).

The invention described herein encompasses screening methods (e.g., assays) for the identification of compounds that affect weight modulation in bovine species. The invention also encompasses agonists and antagonists of bovine MC4-R, including small molecules, large molecules, and antibodies, as well as nucleotide sequences that can be used to inhibit MC4-R gene expression (e.g., antisense and ribozyme molecules), and gene or regulatory sequence replacement constructs designed to enhance MC4-R gene expression (e.g., expression constructs that place the MC4-R gene under the control of a strong promoter system). Such compounds may be used to treat body weight disorders, or to modify body weight, in bovine animals.

In particular, cellular and non-cellular assays are described that can be used to identify compounds that interact with the MC4-R, e.g., modulate the activity of the MC4-R and/or bind to the MC4-R. The cell-based assays can be used to identify compounds or compositions that affect the signal-transduction activity of the MC4-R, whether they bind to the MC4-R or act on intracellular factors involved in the MC4-R signal transduction pathway. Such cell-based assays of the invention utilize cells, cell lines, or engineered cells or cell lines that express the MC4-R. The cells can be further engineered to incorporate a reporter molecule linked to the signal transduced by the activated MC4-R to aid in the identification of compounds that modulate bovine MC4-R signalling activity.

The present invention also encompasses the use of cell-based assays or cell-lysate assays (e.g., in vitro transcription or translation assays) to screen for compounds or compositions that modulate MC4-R gene expression. To this end, constructs containing a reporter sequence linked to a regulatory element of the MC4-R gene can be used in engineered cells, or in cell lysate extracts, to screen for compounds that modulate the expression of the reporter gene product at the level of transcription. For example, such assays could be used to identify compounds that modulate the expression or activity of transcription factors involved in MC4-R gene expression, or to test the activity of triple helix polynucleotides. Alternatively, engineered cells or translation extracts can be used to screen for compounds (including antisense and ribozyme constructs) that modulate the translation of MC4-R mRNA transcripts, and therefore, affect expression of the bovine MC4-R.

The present invention also encompasses MC4-R proteins, biologically active fragments thereof such as peptides and polypeptides, and MC4-R fusion proteins, for use in non-cell based screening assays, generating antibodies, and for diagnostics and therapeutics. As shown in Table 2, the present bovine MC4-R appears to be a serpentine receptor that traverses the membrane seven times, resulting in four extra-cellular domains (ECDs) and four cellular domains (CDs). Peptides corresponding to each ECD, or a polypeptide composed of two or more of the four ECDs linked together, can be engineered as described below. Alternatively, such peptides or polypeptides can be fused to a heterologous protein, e.g., a reporter, an Ig Fc region, etc., to yield a fusion protein. Such peptides, polypeptides, and fusion proteins can be used in the non-cell based assays for screening compounds that interact with, e.g., modulate the activity of the MC4-R and/or bind to the MC4-R.

Bovine MC4-R molecules can be used to treat weight disorders such as obesity, anorexia, or cachexia. Such MC4-R molecules include, but are not limited to, soluble derivatives such as peptides or polypeptides corresponding to one or more MC4-R ECDs, TMDs, or CDs; truncated MC4-R polypeptides lacking one or more ECD, TMD, or CD; and MC4-R fusion protein products (especially MC4-R-Ig fusion proteins, i.e., fusions of the MC4-R or a domain of the MC4-R, to an IgFc domain). Alternatively, antibodies to the MC4-R or anti-idiotypic antibodies that mimic the MC4-R (including Fab fragments), antagonists, or agonists (including compounds that modulate signal transduction which may act on downstream targets in the MC4-R signal transduction pathway) can be used to treat body weight disorders such as obesity, anorexia, or cachexia in bovine species, or to modulate body weight therein.

For example, administration of an effective amount of a soluble MC4-R peptide, polypeptide, MC4-R fusion protein (e.g., MC4-R ECD-IgFc), or anti-idiotypic antibody (or its Fab) that mimics the MC4-R ECD would interact with and thereby “mop up” or “neutralize” endogenous MC4-R ligand, and prevent or reduce binding and receptor activation, leading to weight gain. In yet another approach, nucleotide constructs encoding such MC4-R products can be used to genetically engineer host cells to express such MC4-R products in vivo; these genetically engineered cells can function as “bioreactors” in the body, delivering a continuous supply of the MC4-R, MC4-R peptide, soluble MC4-R polypeptide, or MC4-R fusion protein that will “mop up” or neutralize MC4-R ligand.

“Gene therapy” approaches for the modulation of MC4-R expression and/or activity in the treatment of body weight disorders, or to modify body weight, are within the scope of the present invention. For example, nucleotide constructs encoding functional MC4-Rs, mutant MC4-Rs, as well as antisense and ribozyme molecules, can be used to modulate MC4-R expression.

The invention also encompasses feed and pharmaceutical formulations and methods for treating body weight disorders in bovine animals, or modifying body weight therein.

Investigating The Role of MC4-R in the Regulation of Bovine Body Weight

The specific role of the MC4-R protein in vivo in bovine species can be investigated by engineering MC4-R “knock out” animals in which most of the endogenous MC4-R gene coding sequence is deleted, thereby creating animals that are unable to produce functional MC4-R protein. Unlike MC-R agonist/antagonist studies that are complicated because each of the MC receptors, rather than just MC4-R, can be affected, this specific elimination of only MC4-R function permits evaluation of the biological function of MC4-R.

In order to produce MC4-R knock out animals, MC4-R gene sequences can be utilized to produce bovine MC4-R targeting constructs designed to delete the majority of the bovine MC4-R coding sequence upon homologous recombination with the endogenous bovine MC4-R gene. Embryonic stem (ES) cells containing the disrupted MC4-R gene can be produced, isolated, and microinjected into bovine blastocysts to yield animals chimeric for cells containing a disrupted MC4-R gene. Offspring of the chimeric animals resulting from germline transmission of the ES genome can be obtained, and animals heterozygous for the disrupted MC4-R can be identified.

In order to assess the role of MC4-R in vivo, the animals heterozygous for the MC4-R disrupted gene can be bred together, producing progeny containing wild-type animals, animals heterozygous for the MC4-R mutation, and animals homozygous for the MC4-R mutation. The weight gain of the animals can be monitored regularly. Homozygous null MC4-R mutants are expected to exhibit an increase in weight compared to animals heterozygous for MC4-R deletion and wild-type animals.

Such knock out experiments can provide definitive evidence of the role of MC4-R in bovine weight regulation. The experimental design does not rely on the relationship, if any, of the agouti ligand for the characterization of the functional role of the MC4-R.

Screening Assays for Drugs Useful in the Regulation of Bovine Body Weight

A number of different assay systems can be designed and used to identify compounds or compositions that modulate MC4-R activity or MC4-R gene expression, and therefore, modulate weight control.

The systems described below may be formulated into kits. To this end, the MC4-R or cells expressing the MC4-R can be packaged in a variety of containers, e.g., vials, tubes, microtitre well plates, bottles, and the like. Other reagents can be included in separate containers and provided with the kit, e.g., positive controls samples, negative control samples, melanocortin peptides (including, but not limited to, α-MSH and ACTH derivatives), buffers, cell culture media, etc.

Cell-Based Assays

In accordance with the present invention, a cell-based assay system can be used to screen for compounds that modulate the activity of bovine MC4-Rs, and thereby modulate body weight. To this end, cells that endogenously express MC4-R can be used to screen for compounds. Alternatively, cell lines, such as 293 cells, COS cells, CHO cells, fibroblasts, and the like, genetically engineered to express the MC4-R, can be used for screening purposes. Preferably, host cells genetically engineered to express a functional receptor that responds to activation by melanocortin peptides or other large or small molecules can be used as an endpoint in the assay, e.g., as measured by a chemical, physiological, biological, or phenotypic change, induction of a host cell gene or a reporter gene, change in cAMP or inositol phosphate levels, adenylyl cyclase activity, host cell G protein activity, extracellular acidification rate, host cell kinase activity, proliferation, differentiation, etc.

To be useful in screening assays, the host cells expressing functional bovine MC4-R should give a significant response to MC4-R ligand, preferably greater than two to three-fold induction over background. Host cells should preferably possess a number of characteristics, depending on the readout, to maximize the inductive response by melanocortin peptides or other molecules, for detecting a strong induction of a CRE reporter gene: (a) a low natural level of cAMP or inositol phosphates; (b) G proteins capable of interacting with the MC4-R, including “promiscuous” G proteins (Simon, et al., 1991, Science 252:802-808; Conklin et al., 1993, Nature 363:274-276); (c) a high level of adenylyl cyclase; (d) a high level of protein kinase A; (e) a low level of phosphodiesterases; and (f) a high level of CAMP response element binding protein would be advantageous. To increase response to melanocortin peptide, etc., host cells could be engineered to express a greater amount of favorable factors or a lesser amount of unfavorable factors. In addition, alternative pathways for induction of the CRE reporter could be eliminated to reduce basal levels.

In utilizing such cell systems, the cells expressing a bovine melanocortin receptor are exposed to a test compound or to vehicle controls (e.g., placebos). After exposure, the cells can be assayed to measure the expression and/or activity of components of the signal transduction pathway of the melanocortin receptor, or the activity of the signal transduction pathway itself can be assayed. For example, after exposure, cell lysates can be assayed for induction of cAMP. The ability of a test compound to increase levels of cAMP above those levels seen with cells treated with a vehicle control indicates that the test compound induces signal transduction mediated by the melanocortin receptor expressed by the host cell. When a promiscuous G protein is employed, elevation of inositol phosphate levels can be assayed. In screening for compounds that act as antagonists of bovine MC4-Rs, it is necessary to include ligands that activate the MC4-R, e.g., α-MSH, β-MSH, or ACTH, to test for inhibition of signal transduction by the test compound as compared to vehicle controls.

Constructs containing the CAMP responsive element linked to any of a variety of different reporter genes can be introduced into cells expressing a bovine melanocortin receptor. Numerous reporter genes are known in the art including, but not limited to, chloramphenicol acetyltransferase (CAT), luciferase, GUS, growth hormone, and placental alkaline phosphatase (SEAP). Following exposure of the cells to the test compound, the level of reporter gene expression can be quantitated to determine the test compound's ability to regulate receptor activity. Alkaline phosphatase assays are particularly useful in the practice of the invention as the enzyme is secreted from the cell. Therefore, cell or tissue culture supernatants can be assayed for secreted alkaline phosphatase. In addition, alkaline phosphatase activity can be measured by calorimetric, bioluminescent, or chemilumenscent assays such as those described in Bronstein, I. et al. (1994, Biotechniques 17: 172-177). Such assays provide a simple, sensitive easily automatable detection system for pharmaceutical screening.

When it is desired to discriminate between the melanocortin receptors and to identify compounds that selectively agonize or antagonize the MC4-R, the assays described above can be conducted using a panel of host cells, each genetically engineered to express one of the melanocortin receptors (MC1-R through MC5-R). Host cells can be genetically engineered to express any of the amino acid sequences known for melanocortin receptors 1 through 5 in the art. The cloning and characterization of each receptor has been described: MC1-R and MC2-R (Mountjoy., 1992, Science 257: 1248-1251; Chhajlani & Wikberg, 1992 FEBS Lett. 309: 417-420); MC3-R (Roselli-Rehfuss et al., 1993, Proc. Natl. Acad. Sci. USA 90: 8856-8860; Gantz et al., 1993, J. Biol. Chem. 268: 8246-8250); MC4-R (Gantz et al., 1993, J. Biol. Chem. 268:15174-15179; Mountjoy et al., 1994, Mol. Endo. 8: 1298-1308); and MC5-R (Chhajlani et al., 1993, Biochem. Biophys. Res. Commun. 195: 866-873; Gantz et al., 1994, Biochem. Biophys. Res. Commun. 200; 1234-1220), each of which is incorporated by reference herein in its entirety. Thus, each of the foregoing sequences can be utilized to engineer a cell or cell line that expresses one of the melanocortin receptors for use in screening assays described herein. To identify compounds that specifically or selectively regulate bovine MC4-R activity, the activation, or inhibition of MC4-R activation is compared to the effect of the test compound on the other melanocortin receptors.

Alternatively, if the host cells express more than one melanocortin peptide receptor, the background signal produced by these receptors in response to melanocortin peptides must be “subtracted” from the signal (see Gantz et al., supra). The background response produced by these non-MC4-R melanocortin receptors can be determined by a number of methods, including elimination of MC4-R activity by the use of an antisense nucleic acid, an antibody, or an antagonist. In this regard, it should be noted that wild-type CHO cells demonstrate a small endogenous response to melanocortin peptides which must be subtracted from background. Alternatively, activity contributed from other melanocortin receptors could be eliminated by activating host cells with an MC4-R-specific ligand, or including specific inhibitors of the other melanocortin receptors.

Non-Cell Based Assays

In addition to cell based assays, non-cell based assay systems can be used to identify compounds that interact with, e.g., specifically bind to, MC4-R. Such compounds can act as antagonists or agonists of MC4-R activity, and can be used in the treatment of body weight disorders, or in body weight modification, in bovine animals.

Isolated membranes, for example cellular membranes, for example plasma membranes, can be used to identify compounds that interact with MC4-R. For example, in a typical experiment using isolated membranes, 293 cells can be genetically engineered to express the bovine MC4-R. Membranes can be harvested by standard techniques and used in an in vitro binding assay. For example, ¹²⁵I-labelled ligand (e.g., ¹²⁵I-labelled alpha-MSH, beta-MSH, or ACTH) can bind to the membranes and be assayed for specific activity; specific binding is determined by comparison with binding assays performed in the presence of excess unlabelled ligand.

To identify bovine MC4-R ligands, membranes can be incubated with labelled ligand in the presence or absence of a test compound. Compounds that bind to the receptor and compete with labelled ligand for binding to the membranes reduce the signal compared to vehicle control samples.

Alternatively, soluble MC4-R can be recombinantly expressed and utilized in non-cell based assays to identify compounds that bind to bovine MC4-Rs. The recombinantly-expressed bovine MC4-R molecules, or fusion proteins containing one or more of the ECDs of the bovine MC4-Rs, prepared as described below, can be used in the non-cell based screening assays. Alternatively, peptides corresponding to one or more of the CDs of bovine MC4-Rs, or fusion proteins containing one or more of the CDs of bovine MC4-Rs, can be used in non-cell based assay systems to identify compounds that bind to the cytoplasmic portion of the bovine MC4-Rs; such compounds may be useful to modulate the signal transduction pathway of bovine MC4-Rs. In non-cell based assays, the recombinantly expressed MC4-Rs can be attached to a solid substrate such as a test tube, microtitre well, or a column, by means well known to those in the art (see, for example, Ausubel et al., 1989, Current Protocols in Molecular Biology, John Wiley & Sons, Inc.). The test compounds are then assayed for their ability to bind to bovine MC4-Rs.

In one aspect of the present invention, the screens can be designed to identify compounds that antagonize the interaction between MC4-R and MC4-R ligands such as alpha-MSH, beta-MSH and ACTH. In such screens, the MC4-R ligands are labelled, and the test compounds can be assayed for their ability to antagonize the binding of labelled ligand to MC4-R.

Assays for Compounds or Compositions That Modulate Expression of the MC4-R

In vitro cell-based assays can be designed to screen for compounds that regulate MC4-R expression at either the transcriptional or translational level.

In one embodiment, DNA encoding a reporter molecule can be linked to a regulatory element of a bovine MC4-R gene and used in appropriate intact cells, cell extracts, or lysates to identify compounds that modulate MC4-R gene expression. Appropriate cells or cell extracts are prepared from any cell type that normally expresses an MC4-R gene, thereby ensuring that the cell extracts contain the transcription factors required for in vitro or in vivo transcription. The screen can be used to identify compounds that modulate the expression of the reporter construct. In such screens, the level of reporter gene expression is determined in the presence of the test compound and compared to the level of expression in the absence of the test compound.

To identify compounds that regulate bovine MC4-R translation, cells or in vitro cell lysates containing bovine MC4-R transcripts can be tested for modulation of MC4-R mRNA translation. To assay for inhibitors of MC4-R translation, test compounds are assayed for their ability to modulate the translation of MC4-R mRNA in in vitro translation extracts.

Compounds that decrease the level of MC4-R expression, either at the transcriptional or translational level, may be useful for treatment of body weight disorders in bovine species such as anorexia and cachexia, or to modify body weight in these animals. In contrast, those compounds that increase the expression of MC4-R may be useful to reduce body weight in such animals.

Compounds That Can Be Screened in Accordance With the Invention

The assays described above can identify compounds that affect bovine MC4-R activity. For example, compounds that affect bovine MC4-R activity include, but are not limited to, compounds that bind to bovine MC4-Rs, inhibit binding of the natural ligand, and which either activate signal transduction (agonists) or block activation (antagonists), and compounds that bind to the natural ligand of bovine MC4-Rs and which neutralize ligand activity. Compounds that affect MC4-R gene activity (by affecting MC4-R gene expression, including molecules, e.g., proteins or small organic molecules, that affect transcription or interfere with splicing events so that expression of the full length or the truncated form of the MC4-R can be modulated) can also be identified in the screens of the present invention. However, it should be noted that the assays described can also identify compounds that modulate MC4-R signal transduction (e.g., compounds that affect downstream signalling events, such as inhibitors or enhancers of G protein activities which participate in transducing the signal activated by ligand binding to the MC4-R). The identification and use of such compounds that affect signalling events downstream of MC4-R and thus modulate effects of MC4-R on the development of body weight disorders are within the scope of the invention. In some instances, G protein-coupled receptor response has been observed to subside, or become desensitized, with prolonged exposure to ligand. In an embodiment of the invention, assays can be utilized to identify compounds that block the desensitization of the MC4-R; such compounds may be used to sustain the activity of the MC4-R. Such compounds can be used as part of a therapeutic method for the treatment of body weight disorders, or to modify body weight in otherwise healthy animals.

Compounds that can be screened in accordance with the methods of the present invention include, but are not limited to, peptides, antibodies and fragments thereof, and other organic compounds (e.g., peptidomimetics) that bind to the ECD (or CD) of the MC4-R and which either mimic the activity triggered by the natural ligand (i.e., agonists), or inhibit the activity triggered by the natural ligand (i.e., antagonists), as well as peptides, antibodies or fragments thereof, and other organic compounds that include the ECDs of the MC4-R (or a portion thereof) and that bind to and “neutralize” natural bovine MC4-R ligands.

Compounds include, but are not limited to, peptides such as, for example, soluble peptides, including but not limited to members of random peptide libraries; (see, e.g., Lam, K. S. et al., 1991, Nature 354: 82-84; Houghten, R. et al., 1991, Nature 354: 84-86), and combinatorial chemistry-derived molecular libraries comprising D- and/or L-configuration amino acids, phosphopeptides (including, but not limited to, members of random or partially degenerate, directed phosphopeptide libraries; see, e.g., Songyang, Z. et al., 1993, Cell 72: 767-778), antibodies (including, but not limited to, polyclonal, monoclonal, humanized, anti-idiotypic, chimeric, or single chain antibodies, and Fab, F(ab′)₂, and Fab expression library fragments, and epitope-binding fragments thereof), and small organic or inorganic molecules.

Other compounds that can be screened in accordance with the methods of the present invention include, but are not limited to, small organic molecules that are able to cross the blood-brain barrier, gain entry into an appropriate cell and affect the expression of the MC4-R gene or some other gene involved in the MC4-R signal transduction pathway (e.g., by interacting with the regulatory region or transcription factors involved in gene expression); or such compounds that affect the activity of the MC4-R or the activity of some other intracellular factor involved in the MC4-R signal transduction pathway, such as, for example, the MC4-R associated G protein.

Computer modelling and searching technologies permit identification of compounds, or the improvement of already identified compounds, that can modulate MC4-R expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by determining where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures can be measured with a complexed ligand, natural or artificial, which may increase the accuracy of the active site structure determined.

If an incomplete or insufficiently accurate structure is determined, the methods of computer based numerical modelling can be used to complete the structure or improve its accuracy. Any recognized modelling method can be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, one can identify candidate modulating compounds by searching databases containing compounds along with information on their molecular structure. Such a search seeks compounds having structures that match the determined active site structure and that interact with the groups defining the active site. Such a search can be manual, but is preferably computer assisted. The compounds found from this search are potential bovine MC4-R modulating compounds.

Alternatively, these methods can be used to fashion improved modulating compounds from an already known modulating compound or ligand. The composition of the known compound can be modified and the structural effects of modification can be determined using the experimental and computer modelling methods described above applied to the new composition. The altered structure is then compared to the active site structure of the compound to determine if an improved fit or interaction results. In this manner, systematic variations in composition, such as by varying side chains, can be quickly evaluated to obtain modified modulating compounds or ligands having improved specificity or activity.

Further experimental and computer modelling methods useful in identifying modulating compounds based upon identification of the active sites of MC4-R, and related transduction and transcription factors, will be apparent to those of ordinary skill in the art. Examples of molecular modelling systems are the CHARMm and QUANTA programs (Polygen Corporation, Waltham, Mass.). CHARMm performs the energy minimization and molecular dynamics functions. QUANTA performs the construction, graphic modelling, and analysis of molecular structure. QUANTA allows interactive construction, modification, visualization, and analysis of the behavior of molecules with each other.

A number of authors have reviewed computer modelling of drugs interactive with specific proteins, such as Rotivinen, et al. (1988, Acta Pharmaceutical Fennica 97: 159-166); Ripka (1988, New Scientist 54-57); McKinaly and Rossmann (1989, Annu. Rev. Pharmacol. Toxiciol. 29: 111-122); Perry and Davies (OSAR:Quantitative Structure-Activity Relationships in Drug Design, pp. 189-193, Alan R. Liss, Inc. 1989); Lewis and Dean (1989, Proc. R. Soc. Lond. 236: 125-140 and 141-162); and, with respect to a model receptor for nucleic acid components, Askew, et al. (1989, J. Am. Chem. Soc. 111: 1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario). Although these are primarily designed for application to drugs specific to particular proteins, they can be adapted to design of drugs specific to regions of DNA or RNA, once that region is identified.

Although described above with reference to design and generation of compounds that could alter binding, one could also screen libraries of known compounds, including natural products or synthetic chemicals, and biologically active materials, including proteins, for compounds that are inhibitors or activators.

Compounds identified via assays such as those described herein may be useful, for example, in elaborating the biological function of the bovine MC4-R gene product, for ameliorating body weight disorders in animals, and for modifying body weight therein. Assays for testing the efficacy of compounds identified in the cellular screen can be tested in animal model systems for body weight disorders or modification. Such animal models can be used as test substrates for the identification of drugs, pharmaceuticals, therapies, interventions, and food supplements that may be effective in treating such disorders or modifying body weight. For example, animal models can be administered a compound suspected of exhibiting an ability to ameliorate body weight disorder symptoms or to modify body weight at a sufficient concentration (“effective amount”) and for a time sufficient to elicit such an amelioration of body weight disorder symptoms, or to modify body weight, in the exposed animals. The response of the animals to the exposure can be monitored by assessing the reversal of body weight disorders or modification of body weight. With regard to intervention, any treatments which reverse any aspect of body weight disorder-like symptoms, or that cause a desirable modification of body weight, could be considered as candidates for bovine body weight disorder therapeutic intervention or modification. Dosages of test agents can be determined by deriving dose-response curves, as discussed below.

To this end, transgenic animals that express bovine MC4-R gene products can be used. Animals of any species, including, but not limited to, mice, rats, rabbits, guinea pigs, pigs, micro-pigs, goats, cows, oxen, and non-human primates, e.g., baboons, monkeys, and chimpanzees, can be used to generate MC4-R transgenic animals.

Any technique known in the art can be used to introduce the bovine MC4-R transgene into animals to produce the founder lines of transgenic animals. Such techniques include, but are not limited to pronuclear microinjection (Hoppe, P. C. and Wagner, 1989, U.S. Pat. No. 4,873,191); retrovirus-mediated gene transfer into germ lines (Van der Putten et al., 1985, Proc. Natl. Acad. Sci. USA 82: 6148-6152); gene targeting in embryonic stem cells (Thompson et al., 1989, Cell 56: 313-321); electroporation of embryos (Lo, 1983, Mol Cell. Biol. 3: 1803-1814); and sperm-mediated gene transfer (Lavitrano et al., 1989, Cell 57: 717-723); etc. For a review of such techniques, see Gordon, 1989, Transgenic Animals, Intl. Rev. Cytol. 115: 171-229.

The present invention provides for transgenic animals that carry the MC4-R transgene in all their cells, as well as animals that carry the transgene in some, but not all their cells, i.e., mosaic animals. The transgene can be integrated as a single transgene or in concatamers, e.g., head-to-head tandems or head-to-tail tandems. The transgene can also be selectively introduced into and activated in a particular cell type by following, for example, the teachings of Lasko et al. (1992, Proc. Natl. Acad. Sci. USA 89:6232-6236). The regulatory sequences required for such a cell-type specific activation will depend upon the particular cell type of interest, and will be apparent to those of skill in the art. When it is desired that the bovine MC4-R transgene be integrated into the chromosomal site of the endogenous MC4-R gene,gene targeting is preferred. Briefly, when such a technique is to be utilized, vectors containing nucleotide sequences homologous to the endogenous MC4-R gene and/or sequences flanking the gene are designed for the purpose of integrating, via homologous recombination with chromosomal sequences, into and disrupting the function of the endogenous MC4-R gene. The transgene can also be selectively expressed in a particular cell type with concomitant inactivation of the endogenous MC4-R gene in only that cell type, by following, for example, the teachings of Gu et al. (1994, Science 265:103-106). The regulatory sequences required for such a cell-type specific recombination will depend upon the particular cell type of interest, and will be apparent to those of skill in the art.

Once founder animals have been generated, standard techniques such as Southern blot analysis or PCR techniques can be used to analyze animal tissues to determine whether integration of the transgene has taken place. The level of mRNA expression of the transgene in the tissues of the founder animals can also be assessed using techniques that include, but are not limited, to Northern blot analysis of tissue samples obtained from the animal, in situ hybridization analysis, and RT-PCR. Samples of MC4-R gene-expressing tissue, can also be evaluated immunocyto-chemically using antibodies specific for the MC4-R transgene product.

Bovine MC4-R Proteins, Polypeptides, and Antibodies

Bovine MC4-R proteins, polypeptides, and peptide fragments, mutated, truncated or deleted forms of the MC4-R, and/or MC4-R fusion proteins can be prepared for a variety of uses, including, but not limited to, the generation of antibodies, as reagents in diagnostic assays, the identification of other cellular gene products involved in the regulation or modification of body weight, as reagents in assays for screening for compounds that can be used in the treatment of body weight disorders or to modify body weight, and as pharmaceutical reagents useful in the treatment of body weight disorders related to the MC4-R or in the modification of bovine animal body weight.

Production of Bovine MC4-R Polypeptides

The deduced amino acid sequence of the bovine melanocortin receptor is shown in Table 2, where the seven predicted transmembrane domains (TMD1-7), four extracellular domains (ECD1-4), and four cytoplasmic domains (CD1-4) are also indicated. The serpentine structure of the bovine melanocortin 4 receptor predicts that the hydrophilic domains located between the transmembrane domains are arranged alternately outside and within the cell to form the ECDs of the receptor. Peptides corresponding to one or more domains of the MC4-R (e.g., ECDs, TMDs, or CDs), truncated or deleted bovine MC4-R (e.g., MC4-R in which one or more of the ECDs, TMDs and/or CDs is deleted), as well as fusion proteins in which the full length bovine MC4-R, an MC4-R peptide, or truncated MC4-R is fused to an unrelated protein, are also within the scope of the present invention, as are the nucleotide sequences derived from SEQ ID NO:1 encoding them. Such soluble peptides, polypeptides, proteins, fusion proteins, or antibodies (including anti-idiotypic antibodies) that bind to and “neutralize” circulating natural ligand for the MC4-R, can be used as described in below to effectuate weight gain in animals. To this end, peptides corresponding to individual ECDs of MC4-R, soluble deletion mutants of MC4-R, or the entire MC4-R ECD (engineered by linking the four ECDs together as described below) can be fused to another polypeptide (e.g., an IgFc polypeptide). Fusion of the MC4-R or the MC4-R ECD to an IgFc polypeptide should not only increase the stability of the preparation, but will increase the half-life and activity of the MC4-R-Ig fusion protein in vivo. The Fc region of the Ig portion of the fusion protein can be further modified to reduce immunoglobulin effector function.

Such molecules can be prepared by recombinant DNA techniques. For example, nucleotide sequences encoding one or more of the four domains of the ECD of the serpentine MC4-R can be synthesized or cloned and ligated together to encode a soluble ECD of the MC4-R. The DNA sequence encoding one or more of the four ECDs (ECD1-4 in Table 2) can be ligated together directly or via linker oligonucleotides that encode peptide spacers. Such linkers can encode flexible, glycine-rich amino acid sequences, thereby allowing the domains that are strung together to assume a conformation that can bind MC4-R ligands. Alternatively, nucleotide sequences encoding individual domains within the ECD can be used to express MC4-R peptides.

A variety of host-expression vector systems can be utilized to express nucleotide sequences encoding the appropriate regions of the MC4-R to produce such polypeptides. Where the resulting peptide or polypeptide is a soluble derivative (e.g., peptides corresponding to the ECDs; truncated or deleted in which the TMDs and/or CDs are deleted) the peptide or polypeptide can be recovered from the culture medium. Where the polypeptide or protein is not secreted, the MC4-R product can be recovered from the host cell itself.

The host-expression vector systems also encompass engineered host cells that express the MC4-R or functional equivalents in situ, i.e., anchored in the cell membrane. Purification or enrichment of the MC4-R from such expression systems can be accomplished using appropriate detergents and lipid micelles and methods well known to those skilled in the art. However, such engineered host cells themselves may be used in situations where it is important not only to retain the structural and functional characteristics of the MC4-R, but also to assess biological activity, e.g., in drug screening assays.

The host-expression vector systems that can be used for purposes of the present invention include, but are not limited to, microorganisms such as bacteria (e.g., E. coli, B. subtilis) transformed with recombinant bacteriophage DNA, plasmid DNA, or cosmid DNA expression vectors containing MC4-R nucleotide sequences; yeast (e.g., Saccharomyces, Pichia) transformed with recombinant yeast expression vectors containing the MC4-R nucleotide sequences; insect cell systems infected with recombinant virus expression vectors (e.g., baculovirus) containing the MC4-R sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing MC4-R nucleotide sequences; or mammalian cell systems (e.g., COS, CHO, BHK, 293, or 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Constructs expressing fusion bovine MC4-R proteins, or fusions of bovine MC4-R peptides or polypeptides, are useful in assays to identify compounds that modulate wild-type bovine MC4-R activity. Such fusion constructs include either the entire bovine MC4-R, or fusions comprising the intracellular domain of bovine MC4-R as an in-frame fusion at the carboxy terminus of the GST gene, or the extracellular and transmembrane ligand binding domain of bovine MC4-R fused to GST or an immunoglobulin gene.

In bacterial systems, a number of expression vectors can be advantageously selected depending upon the use intended for the MC4-R gene product being expressed. For example, when a large quantity of such a protein is to be produced for the generation of pharmaceutical compositions of MC4-R protein or for raising antibodies to the MC4-R protein, for example, vectors that direct the expression of high levels of fusion protein products that are readily purified may be desirable. Such vectors include, but are not limited, to the E. coli expression vector pUR278 (Ruther et al., 1983, EMBO J. 2: 1791), in which the MC4-R coding sequence can be ligated individually into the vector in frame with the lacZ coding region so that a fusion protein is produced; pIN vectors (Inouye & Inouye, 1985, Nucleic Acids Res. 13: 3101-3109; Van Heeke & Schuster, 1989, J. Biol. Chem. 264: 5503-5509); and the like. PGEX vectors can also be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

Alternatively, any fusion protein can be readily purified by utilizing an antibody specific for the fusion protein being expressed. For example, a system described by Janknecht et al. allows for the ready purification of non-denatured fusion proteins expressed in human cell lines (Janknecht, et al., 1991, Proc. Natl. Acad. Sci. USA 88: 8972-8976). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺.nitriloacetic acid-agarose columns, and histidine-tagged proteins are selectively eluted with imidazole-containing buffers.

In an insect system, Autographa californica nuclear polyhedrosis virus (AcNPV) is used as a vector to express foreign genes. The virus grows in Spodoptera frugiperda cells. The MC4-R coding sequence can be cloned individually into non-essential regions (for example the polyhedrin gene) of the virus, and placed under control of an AcNPV promoter (for example the polyhedrin promoter). Successful insertion of MC4-R gene coding sequence will result in inactivation of the polyhedrin gene and production of non-occluded recombinant virus (i.e., virus lacking the proteinaceous coat coded for by the polyhedrin gene). The recombinant viruses are then used to infect cells in which the inserted gene is expressed. (e.g., see Smith et al., 1983, J. Virol. 46: 584; Smith, U.S. Pat. No. 4,215,051).

In mammalian host cells, a number of viral-based expression systems can be utilized. In cases where an adenovirus is used as an expression vector, the MC4-R nucleotide sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the MC4-R gene product in infected hosts. (e.g., See Logan & Shenk, 1984, Proc. Natl. Acad. Sci. USA 81: 3655-3659). Specific initiation signals may be required for efficient translation of inserted MC4-R nucleotide sequences. These signals include the ATG initiation codon and adjacent sequences. In cases where an entire MC4-R gene or cDNA, including its own initiation codon and adjacent sequences, is inserted into the appropriate expression vector, no additional translational control signals may be needed. However, in cases where only a portion of the MC4-R coding sequence is inserted, exogenous translational control signals, including, perhaps, the ATG initiation codon, must be provided. Furthermore, the initiation codon must be in frame with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (See Bittner et al., 1987, Methods in Enzymol. 153: 516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. Accordingly, eukaryotic host cells that possess the cellular machinery for proper processing of the primary transcript, glycosylation, and phosphorylation of the gene product can be used. Such mammalian host cells include, but are not limited to, CHO, VERO, BHK, HeLa, COS, MDCK, 293, 3T3, and WI38 cell lines.

For long-term, high-yield production of recombinant proteins, stable expression is preferred. For example, cell lines that stably express the MC4-R sequences described herein can be engineered. Rather than using expression vectors that contain viral origins of replication, host cells can be transformed with DNA controlled by appropriate expression control elements (e.g., promoters, enhancer sequences, transcription terminators, polyadenylation sites, etc.), and a selectable marker. Following the introduction of the foreign DNA, engineered cells can be allowed to grow for 1-2 days in an enriched medium, and then switched to a selective medium. The selectable marker in the recombinant plasmid confers resistance to the selection and allows cells to stably integrate the plasmid into their chromosomes and grow to form foci that in turn can be cloned and expanded into cell lines. This method can advantageously be used to engineer cell lines expressing the MC4-R gene product. Such engineered cell lines can be particularly useful in screening and evaluation of compounds that affect the endogenous activity of the MC4-R gene product.

A number of selection systems can be used, including, but not limited to, the herpes simplex virus thymidine kinase (Wigler, et al., 1977, Cell 11: 223), hypoxanthine-guanine phosphoribosyltransferase (Szybalska & Szybalski, 1962, Proc. Natl. Acad. Sci. USA 48: 2026), and adenine phosphoribosyltransferase (Lowy, et al., 1980, Cell 22:817) genes can be employed in tk⁻, hgprt⁻, or aprt⁻ cells, respectively. Also, antimetabolite resistance can be used as the basis of selection for the following genes: dhfr, which confers resistance to methotrexate (Wigler, et al., 1980, Proc. Natl. Acad. Sci. USA 77: 3567; O'Hare, et al., 1981, Proc. Natl. Acad. Sci. USA 78: 1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150: 1); and hygro, which confers resistance to hygromycin (Santerre, et al., 1984, Gene 30: 147).

Antibodies to MC4-R Molecules

Antibodies that specifically recognize one or more epitopes of bovine MC4-R, or epitopes of conserved variants of MC4-R, or peptide fragments of the MC4-R, are also encompassed by the invention. Generally, the peptides, polypeptides, and proteins of the present invention will, when presented as an immunogen, elicit production of an antibody specifically reactive thereto. Immunoassays for determining binding are well known to those of ordinary skill in the art. Thus, the peptides, polypeptides, and proteins of the present invention can be employed as immunogens for producing antibodies immunoreactive thereto. Such antibodies include, but are not limited to, polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the present invention can be used, for example, in the detection of bovine MC4-Rs in a biological sample and can, therefore, be utilized as part of a diagnostic or prognostic technique whereby animals can be tested for abnormal amounts of MC4-R. Such antibodies can also be utilized in conjunction with, for example, compound screening schemes, as described above, for the evaluation of the effect of test compounds on expression and/or activity of the MC4-R gene product. Additionally, such antibodies can be used in conjunction with the gene therapy techniques described, below, for example, to evaluate the normal and/or engineered MC4-R-expressing cells prior to their introduction into the animal. Such antibodies can additionally be used in a method for the inhibition of abnormal MC4-R activity. Thus, such antibodies can be utilized as part of weight disorder or weight modification treatment methods.

For the production of antibodies, various host animals cam be immunized by injection with the MC4-R, an MC4-R peptide (e.g., one corresponding a functional domain of the receptor, such as an ECD, TMD, or CD), truncated MC4-R polypeptides (MC4-R in which one or more domains, e.g., the TM or CD, has been deleted), functional equivalents of the MC4-R, or mutants of the MC4-R. Such host animals can include, but are not limited to, rabbits, mice, hamsters, and rats, to name but a few. Various adjuvants can be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals. Monoclonal antibodies, which are homogeneous populations of antibodies to a particular antigen, can be obtained by any technique that provides for the production of antibody molecules by continuous cell lines in culture. These include, but are not limited to, the hybridoma technique of Kohler and Milstein, (1975, Nature 256: 495-497; and U.S. Pat. No. 4,376,110), the human B-cell hybridoma technique (Kosbor et al., 1983, Immunology Today 4: 72; Cole et al., 1983, Proc. Natl. Acad. Sci. USA 80: 2026-2030), and the EBV-hybridoma technique (Cole et al., 1985, Monoclonal Antibodies And Cancer Therapy, Alan R. Liss, Inc., pp. 77-96). Such antibodies can be of any immunoglobulin class, including IgG, IgM, IgE, IgA, IgD, and any subclass thereof. The hybridoma producing the mAb of the present invention can be cultivated in vitro or in vivo. Production of high titers of mAbs in vivo makes this the presently preferred method of production.

In addition, techniques developed for the production of “chimeric antibodies” (Morrison et al., 1984, Proc. Natl. Acad. Sci. USA, 81: 6851-6855; Neuberger et al., 1984, Nature, 312: 604-608; Takeda et al., 1985, Nature, 314: 452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird, 1988, Science 242: 423-426; Huston et al., 1988, Proc. Natl. Acad. Sci. USA 85: 5879-5883; and Ward et al., 1989, Nature 334: 544-546) can be adapted to produce single chain antibodies against MC4-R gene products. Single chain antibodies are formed by linking the heavy and light chain fragments of the Fv region via an amino acid bridge, resulting in a single chain polypeptide.

Antibody fragments that recognize specific epitopes can be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries can be constructed (Huse et al., 1989, Science, 246: 1275-1281) to allow rapid and easy identification of monoclonal Fab fragments with the desired specificity.

Antibodies to the MC4-R can, in turn, be utilized to generate anti-idiotype antibodies that “mimic” the MC4-R, using techniques well known to those skilled in the art. (See, e.g., Greenspan & Bona, 1993, FASEB J 7 (5): 437-444; and Nissinoff, 1991, J. Immunol. 147 (8): 2429-2438). For example, antibodies that bind to the MC4-R ECD and competitively inhibit the binding of melanocortins to the MC4-R can be used to generate anti-idiotypes that “mimic” the ECD and, therefore, bind and neutralize melanocortins. Such neutralizing anti-idiotypes or Fab fragments of such anti-idiotypes can be used in therapeutic regimens to neutralize the native ligand and promote weight gain.

Alternatively, antibodies to MC4-R that can act as agonists of MC4-R activity can be generated. Such antibodies will bind to the MC4-R and activate the signal transducing activity of the receptor. Such antibodies would be particularly useful for treating weight disorders such as obesity. In addition, antibodies that act as antagonists of MC4-R activity, i.e., inhibit the activation of MC4-R, can be used to treat weight disorders such as anorexia or cachexia, or to increase body weight over normal levels.

Screening peptides or polypeptides for specific binding to antibodies or antibody fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids in length, and often from about 8 to 15 amino acids in length. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vectors, and screening kits are commercially available from suppliers such as Invitrogen (Carlsbad, Calif.).

Gene Therapy Approaches to Controlling MC4-R Activity and Regulating or Modifying Body Weight

The expression of MC4-R can be controlled in vivo (e.g., at the transcriptional or translational level) using gene therapy approaches to regulate MC4-R activity and treat body weight disorders, or modify body weight. Certain approaches are described below.

Gene Replacement Therapy

With respect to an increase in the level of normal MC4-R gene expression and/or MC4-R gene product activity, MC4-R nucleic acid sequences can be utilized for the treatment of body weight disorders, including obesity. Where the cause of obesity is a defective MC4-R gene, treatment can be administered, for example, in the form of gene replacement therapy. Specifically, one or more copies of a normal MC4-R gene or a portion of the MC4-R gene that directs the production of an MC4-R gene product exhibiting normal function, can be inserted into the appropriate cells within an animal subject, using vectors that include, but are not limited to, adenovirus, adeno-associated virus, retrovirus, and herpes virus vectors, in addition to other particles that introduce DNA into cells, such as liposomes.

Because the MC4-R gene is expressed in the brain, including the cortex, thalamus, brain stem, and spinal cord and hypothalamus, such gene replacement therapy techniques should be capable of delivering MC4-R gene sequences to these cell types within animals. Thus, the techniques for delivery of the MC4-R gene sequences should be designed to readily cross the blood-brain barrier, which are well known to those of skill in the art (see, e.g., PCT International Publication No. WO 89/10134, or, alternatively, should involve direct administration of such MC4-R gene sequences to the site of the cells in which the MC4-R gene sequences are to be expressed.

Alternatively, targeted homologous recombination can be utilized to correct the defective endogenous MC4-R gene in the appropriate tissue; e.g., brain tissue. In animals, targeted homologous recombination can be used to correct or modify the defect in ES cells in order to generate offspring with a corrected or modified trait.

Additional methods that can be utilized to increase the overall level of MC4-R gene expression and/or MC4-R activity include the introduction of appropriate MC4-R-expressing cells, preferably autologous cells, into an animal at positions and in numbers sufficient to ameliorate the symptoms of body weight disorders, including obesity. Such cells can be either recombinant or non-recombinant. Among the cells that can be administered to increase the overall level of MC4-R gene expression in an animal are normal cells, or hypothalamus cells that express the MC4-R gene. The cells can be administered at the anatomical site in the brain, or as part of a tissue graft located at a different site in the body. Such cell-based gene therapy techniques are well known to those skilled in the art. See, e.g., Anderson, et al., U.S. Pat. No. 5,399,349; Mulligan & Wilson, U.S. Pat. No. 5,460,959.

Finally, compounds identified in the assays described above that stimulate or enhance the signal transduced by activated MC4-R, e.g., by activating downstream signalling proteins in the MC4-R cascade and thereby by-passing the defective MC4-R, can be used to achieve weight loss. The formulation and mode of administration of such compounds will depend upon the physico-chemical properties of the compound. The administration should include known techniques that facilitate crossing of the blood-brain barrier.

Inhibition of MC4-R Expression in Animals

In an alternate embodiment, weight gain treatment can be designed to reduce the level of endogenous MC4-R gene expression, e.g., using antisense or ribozyme approaches to inhibit or prevent translation of MC4-R mRNA transcripts; triple helix approaches to inhibit transcription of the MC4-R gene; or targeted homologous recombination to inactivate or “knock out” the MC4-R gene or its endogenous promoter. Such gene therapy can be utilized where the inhibition of MC4-R expression is designed to increase animal body weight Because the MC4-R gene is expressed in the brain, delivery techniques should be preferably designed to cross the blood-brain barrier (see PCT International Publication WO 89/10134). Alternatively, the antisense, ribozyme, or DNA constructs described herein can be administered directly to the site containing the target cells.

Antisense approaches involve the design of oligonucleotides (either DNA or RNA) that are complementary to mRNA. The antisense oligonucleotides will bind to the complementary mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

While antisense nucleotides complementary to the coding region sequence could be used, those complementary to the transcribed untranslated region are most preferred (see Table 2). Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have also been shown to be effective in inhibiting translation of mRNAs. See, generally, Wagner, R., 1994, Nature 372: 333-335. Thus, oligonucleotides complementary to either the 5′- or 3′-non-translated, non-coding regions of MC4-R could be used in an antisense approach to inhibit translation of endogenous mRNA. Oligonucleotides complementary to the 5′ untranslated region of the mRNA should include the complement of the AUG start codon. Antisense oligonucleotides complementary to mRNA coding regions are less efficient inhibitors of translation, but could be used in accordance with the invention. Whether designed to hybridize to the 5′-, 3′-, or coding region of MC4-R mRNA, antisense nucleic acids should be at least about six nucleotides in length, more preferably from about 6 to about 50 nucleotides in length, and even more preferably, at least about 10 nucleotides, at least about 17 nucleotides, at least about 25 nucleotides, or at least about 50 nucleotides in length. About 18 to about 22 nucleotides in length is preferred.

Regardless of the choice of target sequence, it is preferred that in vitro studies first be performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide be compared with those obtained using a control oligonucleotide. Preferably, the control oligonucleotide is of approximately the same length as the test oligonucleotide, and that the nucleotide sequence of the oligonucleotide differs from the antisense sequence no more than is necessary to prevent specific hybridization to the target sequence.

The oligonucleotides can be DNA, RNA, or chimeric mixtures, derivatives, or modified versions thereof, single-stranded or double-stranded. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. USA 84:648-652; PCT Publication No. WO 88/09810, published Dec. 15, 1988) or the blood-brain barrier (see, e.g., PCT Publication No. WO 89/10134, published Apr. 25, 1988), or hybridization-triggered cleavage agents. (See, e.g., Krol et al., 1988, BioTechniques 6: 958-976) or intercalating agents. (See, e.g., Zon, 1988, Pharm. Res. 5: 539-549). To this end, the oligonucleotide can be conjugated to another molecule, e.g., a peptide, hybridization triggered cross-linking agent, transport agent, hybridization-triggered cleavage agent, etc.

The antisense oligonucleotide can comprise at least one modified base moiety which can be selected from the group including, but not limited to, 5-fluorouracil, 5-bromo-uracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethyl-aminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methyl-guanine, 3-methylcytosine, 5-methyl-cytosine, N6-adenine, 7-methylguanine, 5-methylamino-methyluracil, 5-methoxy-aminomethyl-2-thiouracil, beta-D-mannosylqueosine, 5′-methoxycarboxymethyluracil, 5-methoxyuracil, 2-methyl-thio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide can also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an alpha-anomeric oligonucleotide. An alpha-anomeric oligonucleotide forms specific double-stranded hybrids with complementary RNA in which, contrary to the usual beta-units, the strands run parallel to each other (Gautier et al., 1987, Nucl. Acids Res. 15: 6625-6641). The oligonucleotide is a 2′-O-methylribonucleotide (Inoue et al., 1987, Nucl. Acids Res. 15: 6131-6148), or a chimeric RNA-DNA analogue (Inoue et al., 1987, FEBS Lett. 215: 327-330).

Oligonucleotides of the invention can be synthesized by standard methods known in the art, e.g., by use of an automated DNA synthesizer (such as are commercially available from Biosearch, Applied Biosystems, etc.). As examples, phosphorothioate oligonucleotides can be synthesized by the method of Stein et al. (1988, Nucl. Acids Res. 16: 3209), and methylphosphonate oligonucleotides can be prepared by use of controlled pore glass polymer supports (Sarin et al., 1988, Proc. Natl. Acad. Sci. U.S.A. 85: 7448-7451), etc.

The antisense molecules should be delivered to cells that express the MC4-R in vivo, e.g., neural tissue. A number of methods have been developed for delivering antisense DNA or RNA to cells. For example, antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

However, it is often difficult to achieve intracellular concentrations of the antisense molecules sufficient to suppress translation of endogenous mRNAs. Therefore, a preferred approach utilizes a recombinant DNA construct in which the antisense oligonucleotide is placed under the control of a strong pol III or pol II promoter. The use of such a construct to transfect target cells in the animal will result in the transcription of sufficient amounts of single stranded RNAs that will form complementary base pairs with the endogenous MC4-R transcripts and thereby prevent translation of the MC4-R mRNA. For example, a vector can be introduced in vivo such that it is taken up by a cell and directs the transcription of an antisense RNA. Such a vector can remain episomal, or become chromosomally integrated, as long as it can be transcribed to produce the desired antisense RNA. Such vectors can be constructed by recombinant DNA methods standard in the art. Vectors can be plasmid, viral, or others known in the art, used for replication and expression in mammalian cells. Expression of the sequence encoding the antisense RNA can be by any promoter known in the art to act in mammalian, preferably bovine cells. Such promoters can be inducible or constitutive. Such promoters include, but are not limited to, the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc. Any type of plasmid, cosmid, YAC, or viral vector can be used to prepare the recombinant DNA construct that can be introduced directly into the tissue site; e.g., the choroid plexus or hypothalamus. Alternatively, viral vectors that selectively infect the desired tissue can be used. For example, for brain, herpesvirus vectors can be used, in which case administration can be accomplished by another route (e.g., systemically).

Ribozyme molecules designed to catalytically cleave MC4-R mRNA transcripts can also be used to prevent translation of bovine MC4-R mRNA and expression of MC4-R. (See, e.g., PCT International Publication WO 90/11364, published Oct. 4, 1990; Sarver et al., 1990, Science 247: 1222-1225). While ribozymes that cleave mRNA at site-specific recognition sequences can be used to destroy MC4-R mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art, and is described more fully in Haseloff and Gerlach, 1988, Nature, 334: 585-591. There are hundreds of potential hammerhead ribozyme cleavage sites within the nucleotide sequence of bovine MC4-R cDNA (see SEQ ID NO:1). Preferably, the ribozyme is engineered so that the cleavage recognition site is located near the 5′ end of the MC4-R mRNA, i.e., to increase efficiency and minimize the intracellular accumulation of non-functional mRNA transcripts.

The ribozymes of the present invention also include RNA endoribonucleases (hereinafter “Cech-type ribozymes”) such as the one that occurs naturally in Tetrahymena thermophila (known as the IVS, or L-19 IVS RNA), and which has been extensively described by Thomas Cech and collaborators (Zaug, et al., 1984, Science, 224: 574-578; Zaug and Cech, 1986, Science, 231: 470-475; Zaug, et al., 1986, Nature, 324: 429-433; PCT International Publication WO 88/04300; Been and Cech, 1986, Cell, 47: 207-216). The Cech-type ribozymes have an eight base pair active site that hybridizes to a target RNA sequence, whereafter cleavage of the target RNA takes place. The invention encompasses those Cech-type ribozymes that target eight base-pair active site sequences present in bovine MC4-Rs.

As in the antisense approach, the ribozymes can be composed of modified oligonucleotides (e.g., for improved stability, targeting, etc.), and should be delivered to cells that express the MC4-R in vivo, e.g., the hypothalamus. A preferred method of delivery involves using a DNA construct “encoding” the ribozyme under the control of a strong constitutive pol III or po1 II promoter so that transfected cells will produce sufficient quantities of the ribozyme to destroy endogenous MC4-R messages and inhibit translation. Because ribozymes, unlike antisense molecules, are catalytic, a lower intracellular concentration is required for efficiency.

Endogenous MC4-R gene expression can also be reduced by inactivating or “knocking out” the MC4-R gene or its promoter using targeted homologous recombination (e.g., see Smithies et al., 1985, Nature 317:230-234; Thomas & Capecchi, 1987, Cell 51: 503-512; Thompson et al., 1989 Cell 5: 313-321). For example, a mutant, non-functional MC4-R (or a completely unrelated DNA sequence) flanked by DNA homologous to the endogenous MC4-R gene can be used, with or without a selectable marker and/or a negative selectable marker, to transfect cells that express MC4-R in vivo. Insertion of the DNA construct, via targeted homologous recombination, results in inactivation of the MC4-R gene. Such approaches are particularly suited in the agricultural field where modifications to ES (embryonic stem) cells can be used to generate animal offspring with an inactive MC4-R (e.g., see Thomas & Capecchi, 1987, and Thompson, 1989, supra). This approach can be adapted for use in humans provided the recombinant DNA constructs are directly administered or targeted to the required site in vivo using appropriate viral vectors, e.g., herpes virus vectors for delivery to brain tissue, e.g., the hypothalamus and/or choroid plexus.

Alternatively, endogenous MC4-R gene expression can be reduced by targeting deoxyribonucleotide sequences complementary to the regulatory region of the MC4-R gene (i.e., the MC4-R promoter and/or enhancers) to form triple helical structures that prevent transcription of the MC4-R gene in target cells in the body. (See generally, Helene, C. 1991, Anticancer Drug Des., 6 (6): 569-84; Helene, C., et al., 1992, Ann. N.Y. Acad. Sci., 660: 27-36; and Maher, L. J., 1992, Bioassays 14 (12): 807-15).

Delivery of Soluble Bovine MC4-R Polypeptides

Genetically engineered cells that express soluble bovine MC4-R ECDs or fusion proteins, e.g., fusion Ig molecules, can be administered in vivo where they can function as “bioreactors” that deliver a supply of the soluble molecules. Such soluble MC4-R polypeptides and fusion proteins, when expressed at appropriate concentrations, should neutralize or “mop up” the native ligand for MC4-R, and thus act as inhibitors of MC4-R activity, inducing weight gain.

Pharmaceutical Formulations and Methods of Treating Body Weight Disorders and Modifying Body Weight in Animals

The present invention encompasses methods and compositions for modifying body weight in animals. The present invention also encompasses methods useful in treating body weight disorders in animals, including but not limited to, obesity, cachexia, and anorexia. Because a loss of normal MC4-R gene product function results in the development of an obese phenotype, an increase in MC4-R gene product activity, or activation of the MC4-R pathway (e.g., downstream activation), would facilitate progress towards a normal body weight state in obese animals exhibiting a deficient level of MC4-R gene expression and/or MC4-R activity.

Alternatively, treatment of symptoms of certain body weight disorders such as, for example, cachexia, which involve a lower than normal body weight phenotype, or the need or desire to increase bovine animal body weight, can be achieved by decreasing the level of MC4-R gene expression and/or MC4-R gene activity, and/or downregulating activity of the MC4-R pathway (e.g., by targeting downstream signalling events). Different approaches for achieving these effects are discussed below.

Agonists of MC4-R can be used to induce weight loss in animals. Antagonists of MC4-R activity can be used to induce weight gain, and to treat conditions such as anorexia or cachexia. It is not necessary that the compound demonstrate absolute specificity for the MC4-R. For example, compounds that agonize both MC4-R and MC4-R could be used; such compounds could be administered so that delivery to the brain is optimized to achieve weight reduction, and side effects, such as peripheral melanin production, may well be tolerated. Compounds that do not demonstrate a specificity for MC4-R can be administered in conjunction with another therapy or drug to control the side-effects that may result from modulating another melanocortin receptor; however, compounds that demonstrate a preference or selectivity for MC4-R over MC3-R are preferred since both receptors are expressed in the brain where localized delivery cannot be used to compensate for lack of receptor specificity.

Dose Determinations

Toxicity and therapeutic efficacy of such compounds can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index, and can be expressed as the ratio LD₅₀/ED₅₀. Compounds that exhibit large therapeutic indices, for example 20:1, are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in bovine animals. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in animals. Levels in plasma can be measured, for example, by high performance liquid chromatography.

Formulations and Use

Pharmaceutical compositions for use in accordance with the present invention can be formulated in conventional manner using one or more physiologically acceptable carriers, diluents, or excipients used in veterinary medicine.

Thus, the compounds and their physiologically acceptable salts and solvates can be formulated for administration by inhalation or insufflation (either through the mouth or the nose), or oral, buccal, parenteral, or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl- or propyl-p-hydroxybenzoates or sorbic acid). The preparations can also contain buffer salts, flavoring, coloring, and sweetening agents as appropriate.

Preparations for oral administration can be suitably formulated to give controlled release of the active compound.

Preparations for oral administration can be mixed with standard animal feed.

For buccal administration, the compositions can take the form of tablets or lozenges formulated in conventional manner.

For administration by inhalation, the compounds for use according to the present invention can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount (metered dose inhaler). Capsules and cartridges of, e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules, pre-filled syringes, or in multi-dose containers, with an added preservative. The compositions can take such forms as suspensions, solutions, or emulsions in oily or aqueous vehicles, and can contain formulatory agents such as suspending, stabilizing, and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described above, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device which can contain one or more unit dosage forms containing the active ingredient. The pack can, for example, comprise metal or plastic foil, such as a blister pack. The pack or dispenser device can be accompanied by instructions for administration.

The following examples are provided to illustrate various aspects of the present invention, and should not be construed to be limiting thereof in any way.

### EXAMPLE 1

### Cloning of Bovine Melanocortin 4 Receptor cDNA by Polymerase Chain Reaction and cDNA Library Screening

Cloning of the bovine MC4 receptor was carried out using two oligonucleotide primers (MC4-A: SEQ ID NO:3; and MC4-B: SEQ ID NO:4):

| | |
|---|---|
| Primer MC4-A | |
| 5′-TTTTTGTCTCTCCTGAGGTGTTTGTGACTCT-3′ | (SEQ ID NO:3) |
| Primer MC4-B | |
| 3′-AGAGATACAGGTGTACAAGGACTACCGGTCC-5′ | (SEQ ID NO:4) |

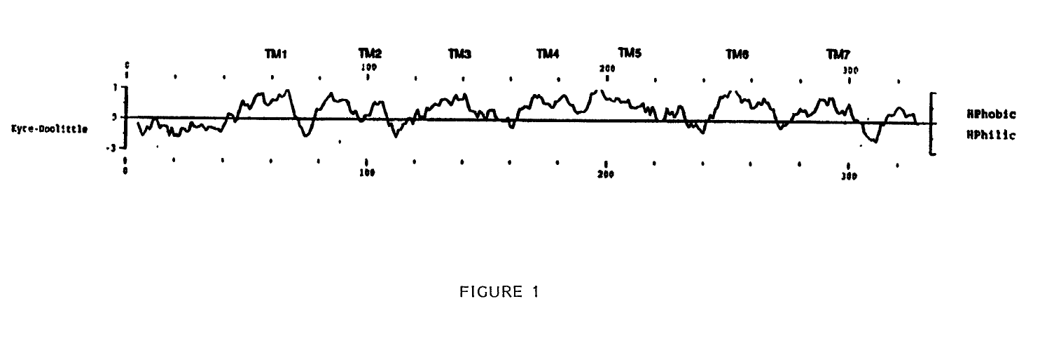

based on the homologous region of the known human and rat MC4-R cDNA sequences (Mountjoy, K. G. et al., 1994, Mol. Endocrinol. 8:1298-1308) (FIG. 1). These primers were used to amplify the MC4-R cDNA fragment from bovine brain cDNA (Clontech Quick Clone cDNA kit; Clontech, Palo Alto, Calif.; catalog no. 7096-1) as template in a polymerase chain reaction experiment.

The polymerase chain reaction was carried out as follows: Ten pmoles each of primers MC4-A (SEQ ID NO:3) and MC4-B (SEQ ID NO:4) were added to the PCR reaction mixture (Clontech Advantage cDNA PCR Kit; catalog no. K1905-1) containing 2 ng of bovine brain cDNA (Clontech) and 0.2 mM each of DATP, dCTP, dTTP and dGTP, according to the supplier's instructions. The reaction mixture was preheated at 94° C. for 1 min., and cycled at 94° C./30 sec, 55° C./1 min., and 68° C./1 min., for 35 cycles, followed by heating the mixture at 68° C. for an additional 5 min. and cooling to 4° C. The resulting bovine cDNA fragment (about 1 μg) was made blunt-ended using T4 DNA polymerase (Gibco-BRL, catalog no. 18005-017), and the fragment was phosphorylated using T4-DNA kinase (Gibco-BRL, catalog no. 18004-010), under reaction conditions recommended by the manufacturer. The bovine cDNA fragment was then purified by 1% agarose gel electrophoresis, and the purified fragment was cloned into pUC18 (Pharmacia Biotech, Milwaukee, Wis., catalog no. 27-5266-01) at the SmaI site, producing plasmid pUC18/bMC4R. Plasmid pUC18/bMC4R was sequenced using PE-Biosystems automated sequencing equipment, and the cloned bovine MC4-R fragment in the plasmid was confirmed to be a genuine 570 bp long bMC4-R cDNA, having 88.5% identity to the human MC4-R cDNA sequence within the same region.

A radioactively labeled 570 bp bovine MC4-R cDNA probe was prepared by PCR for screening of a bovine brain lambda cDNA library (Stratagene, La Jolla, Calif., catalog no. 937719). The probe was prepared by using 1 ng of plasmid pUC18/bMC4R as the template and 20 pmoles of each primer (MC4-A, SEQ ID NO:3; and MC4-B, SEQ ID NO:4) in a PCR experiment. In the PCR experiment, 600 pmoles each of DATP, dGTP, and dTTP, together with 15 μl of [α-³²P]-dCTP (specific activity 3000 Ci/mmol, 10 mCi/ml; New England Nuclear Inc., Boston Mass., catalog no. BLU513H), were used in a reaction with 0.5 μl of Takara LA Taq DNA polymerase (95 units/μl; Takara Shozo Co., Shiga, Japan, Code no. RR002M). The PCR was performed at 95° C./30 sec, 57° C./1 min., and 72° C./1 min. for 30 cycles, and then kept at 4° C. after the reaction cycles. The labeled 570 bp cDNA probe was purified using a Sephadex G-50 Quick Spin Column (Boehringer-Mannheim, Indianapolis, Ind., catalog no. 1-273-965). The labeled cDNA probe was used to screen a bovine brain lambda cDNA Uni-ZAP XR library (Stratagene, La Jolla, Calif., catalog no. 937719) according to the supplier's instructions. One positive bovine MC4-R cDNA clone having a 1.9 kb size insert was discovered by screening 1.25×10⁶ plaque forming units (pfu) in the library. The bovine MC4-R cDNA was excised in vivo from the Uni-ZAP vector using Stratagene's Exassist/Sola System according to the supplier's instructions (Stratagene, La Jolla, Calif., catalog no. 937719). The excised bovine MC4-R cDNA was recircularized to form the phagemid pBluescript-bMC4-R. This phagemid was sequenced, and found to contain a full-length bovine MC4-R cDNA having the following sequence (SEQ ID NO:1):

| | |
|---|---|
| 1 | GGAGCTCCAC CGCGGTGGCG GCCGCTCTAG AACTAGTGGA |

TCCCCCGGGC

| | |
|---|---|
| 51 | TGCAGGAATT CGGCACGAGC AGCCTAAGAT TTCCAAGTGA |

TGCTGACCAG

| | |
|---|---|
| 101 | AGCCACACTT GAAAGAGACT GAAAACTTCC TTTCCAGCTC |

CGGAGCATGG

| | |
|---|---|
| 151 | GACATTTATT CACAGCAGGC ATGCCACTCT CCGCCGCCTA |

ACTTTCGTTT

| | |
|---|---|
| 201 | GGGGCAAGTC AAGACTGGAG AAAGGTGCTG AGGCTGCCAG |

ATCCAGGAGG

| | |
|---|---|
| 251 | TTCAGTCAGT CCAGAGGGGA CCTGAATCCA AAATGAACTC |

TACCCAGCCC

| | |
|---|---|
| 301 | CTTGGGATGC ACACCTCTCT CCACTCCTGG AACCGCAGCG |

CCCACGGAAT

| | |
|---|---|
| 351 | GCCCACCAAT GTCAGTGAGT CCCTGGCAAA AGGCTACTCG |

GACGGGGGGT

| | |
|---|---|
| 401 | GCTATGAGCA GCTCTTTGTC TCTCCCGAGG TGTTTGTGAC |

TCTGGGGGTC

| | |
|---|---|
| 451 | ATCAGCTTGT TGGAGAATAT TCTGGTGATC GTGGCCATAG |

CCAAGAACAA

| | |
|---|---|
| 501 | GAATCTGCAC TCACCCATGT ACTTTTTCAT CTGCAGCCTG |

GCTGTGGCTG

| | |
|---|---|
| 551 | ACATGTTGGT GAGCGTTTCC AACGGGTCGG AAACCATTGT |

CATCACCCTG

| | |
|---|---|
| 601 | CTGAACAGCA CGGACACGGA CGCGCAGAGC TTCACGGTGG |

ATATTGACAA

| | |
|---|---|
| 651 | TGTCATTGAC TCGGTGATCT GTAGCTCCTT GCTTGCCTCC |

ATCTGCAGCT

| | |
|---|---|
| 701 | TGCTGTCGAT CGCGGTGGAC AGGTACTTCA CTATCTTCTA |

TGCGCTCCAG

| | |
|---|---|
| 751 | TACCATAACA TCATGACGGT GAAGCGGGTG GCGATCACCA |

TCAGCGCCAT

| | |
|---|---|
| 801 | CTGGGCAGCC TGCACGGTGT CGGGCGTCTT GTTCATCATT |

TACTCAGACA

| | |
|---|---|
| 851 | GCAGTGCTGT TATCATCTGC CTCATCACCG TGTTCTTCAC |

CATGCTGGCT

| | |
|---|---|
| 901 | CTCATGGCGT CTCTCTATGT CCACATGTTC CTCATGGCCA |

GACTCCACAT

| | |
|---|---|
| 951 | TAAGAGGATC GCGGTCCTGC CAGGTAGCGG CACCATCCGC |

CAGGGCGCCA

| | |
|---|---|
| 1001 | ACATGAAGGG GGCGATTACC CTGACCATAC TGATCGGGGT |

CTTTGTTGTC

| | |
|---|---|
| 1051 | TCCTGGGCCC CCTTCTTCCT GCACCTGATA TTCTACATCT |

CTTGTCCCCA

| | |
|---|---|
| 1101 | GAACCCATAC TGTGTGTGTT TCATGTCTCA CTTTAACCTG |

TACCTCATCC

| | |
|---|---|
| 1151 | TCATCATGTG CAATTCCATC ATTGACCCTC TGATTTATGC |

CCTGCGGAGC

| | |
|---|---|
| 1201 | CAAGAACTGA GGAAAACCTT CAAACAGATC ATTTGTTGCT |

CTCCTCTAGG

| | |
|---|---|
| 1251 | TGGCCTCTGT GATTTGTCTA GCAGATATTA`AATGGGGACA |

AACGCGATGC

| | |
|---|---|
| 1301 | TAAACACAAG CTTAAGAGAC TTTCTCCTTC TCATATGTAC |

AACCTGAACA

| | |
|---|---|
| 1351 | GTCTGTATCA GCCACAGCTT TTTCTTCTGT GTACGGCATG |

GAGTGAAAAT

| | |
|---|---|
| 1401 | TTCTATTGTA TCAGTTGAAG TTTGTGATTT TTTTCTGATG |

TGAAACAGTG

| | |
|---|---|
| 1451 | CCCAGTCTTG GTGTATTTTT AATGTCATGC TACTTTCTGG |

CTGTAAAATG

| | |
|---|---|
| 1501 | TGAATCCACA TCACAGGTTA TAGGCACTAT GCATTTATAA |

AAAAAGAAGA

| | |
|---|---|
| 1551 | AAAAAAGTCC TTATGAGGAG TTTAACAGTG TTTCCTTCTT |

GTTATTTACA

| | |
|---|---|
| 1601 | AGGATGTGAC ACTTTGCTTG CTTTTGTAAC ATGGAAATCA |

CAGCTTCATT

| | |
|---|---|
| 1651 | AAGTATATCC TCATAAGTGG TTTTTTTATG TTATACTTTA |

CAACACTGAA

| | |
|---|---|
| 1701 | GTGTAAAAAT TTGATTCTAG CATTTAGGGG AGAAATATTG |

AGAACATATT

| | |
|---|---|
| 1751 | GCTTAATCAT AAAAAACAAG CTGAAATTTC AGGTAATTTA |

ATAAGACTTT

| | |
|---|---|
| 1801 | CTCATTCATT CTTCCTGTGC AGAAGTTGAA ATGAAGCTTG |

TATTGGGAGA

| | |
|---|---|
| 1851 | AAAACAGTTA CTTAAAAAAA AAAAAAAAAA ACTCGAGGGG |

GGGCCCGGTA

| | |
|---|---|
| 1901 | CCCAATTCGC CCTATAGTGA GTCGTATTAC AATTCACTGG |

CCGTCGTTTT

The foregoing cDNA molecule contains 1950 nucleotides, with an open reading frame from nucleotide 283 to nucleotide 1278, with a TAA termination codon at nucleotides 1279 to 1281. This open reading frame encodes a bovine MC4-R protein 332 amino acids in length having the following deduced amino acid sequence (SEQ ID NO:2):

MNSTQPLGNHTSLHSWNRSAHGMPTNVSESLAKGYSDGGCYEQLFVSPEV
FVTLGVISLLENILVTVATAKNKNLHSPNYFFICSLAVADMLVSVSNGSE
TIVITLLNSTDTDAQSFTVDIDNVIDSVICSSLLASICSLLSIAVDRYFT
TFYALQYHNIMTVKRVAITISAIWAACTVSGVLFTIYSDSSAVIICLITV
FFTMLALMASLYVHMFLMARLHTKRTAVLPGSGTTRQGANMKGAITLTIL
TGVFVVCWAPFFLHLIFYTSCPQNPYCVCFMSHFNLYLTLIMCNSITDPL
IYALRSQELRKTFKEIICCSPLGGLCDLSSRY

As shown in Table 2, the bovine MC4-R protein sequence deduced from the cDNA sequence is 92.5% identical to the human MC4-R protein sequence. As shown Table 2 and FIG. 1, the bovine MC4-R protein is a typical G-protein coupled receptor containing seven putative transmembrane domains, four putative extracellular domains, and four putative cellullar domains, having amino acid sequences as follows:

MNSTQPLGMHTSLHSWNRSAHGMPTNVSESLAKGYSDGGCYEQ (SEQ ID NO:5: putative bovine MC4-R ECD 1 amino acid sequence);

LFVSPEVFVTLGVISLLENILVIVAI (SEQ ID NO:6: putative bovine MC4-R TMD 1 amino acid sequence);

AKNKNLHSP (SEQ ID NO:7: putative bovine MC4-R CD 1 amino acid sequence);

MYFFICSLAVADMLVSVSNGSETIVITLLN (SEQ ID NO:8: putative bovine MC4-R TMD 2 amino acid sequence);

STDTDAQSFTVDIDNVIDS (SEQ ID NO:9: putative bovine MC4-R ECD 2 amino acid sequence);

VICSSLLASICSLLSIAVDRYFTIFYAL (SEQ ID NO:10: putative bovine MC4-R TMD 3 amino acid sequence);

QYHNIMTVKR (SEQ ID NO:11: putative bovine MC4-R CD 2 amino acid sequence);

VAITISAIWAACTVSGVLFIIY (SEQ ID NO:12: putative bovine MC4-R TMD 4 amino acid sequence);

SDSSA (SEQ ID NO:13: putative bovine MC4-R ECD 3 amino acid sequence);

VIICLITVFFTMLALMASLYVHMFLMA (SEQ ID NO:14: putative bovine MC4-R TMD 5 amino acid sequence);

RLHIKRIAVLPGSGTIRQ (SEQ ID NO:15: putative bovine MC4-R CD 3 amino acid sequence);

GANMKGAITLTILIGVFVVCWAPFFLHLIF (SEQ ID NO:16: putative bovine MC4-R TMD 6 amino acid sequence);

YISCP (SEQ ID NO:17: putative bovine MC4-R ECD 4 amino acid sequence);

QNPYCVCFMSHFNLYLILIMCNSIIDPLIYAL (SEQ ID NO:18: putative bovine MC4-R TMD 7 amino acid sequence);

RSQELRKTFKEIICCSPLGGLCDLSSRY (SEQ ID NO:19: putative bovine MC4-R CD 4 amino acid sequence).

The 332 amino acid bovine MC4-R protein sequence contains a putative N-terminal signal peptide sequence that directs the protein to the cell membrane.

### EXAMPLE 2

### Functional Expression of Bovine MC4-R cDNA in Human Embryonic Kidney 293 Cells

The phagemid pBluescript-bMC4-R (10 μg) containing a full-length (1.9 kb) bovine MC4-R cDNA (SEQ ID NO:1) was digested using restriction endonucleases EcoRI (20 units, Gibco-BRL) and XhoI (20 units, Gibco-BRL) at 37° C. for 1 hr following the supplier's instructions. The resulting 1.9 kb bovine MC4-R cDNA fragment was then subcloned into the mammalian cell expression plasmid pcDNA3.1(+) (Invitrogen, Carlsband, Calif.) following standard cloning procedures (Sambrook et al., 1989, Molecular Cloning, A Laboratory Manual, Second Edition, Cold Spring Harbor Laboratory Press). The resulting bovine MC4-R expression plasmid, pcDNA3.1-bMC4R, was then used in conjunction with a reporter plasmid, pGL2-promoter/CRE (cyclic AMP activated transcription binding protein response element), to transiently co-transfect human embryonic kidney 293 cells (ATCC CRL 1573) using the LipofectAMINE PLUS reagent kit (Gibco-BRL, catalog no. 10964-013) following the supplier's instructions.

The pGL2-promoter/CRE plasmid, containing tandem cAMP activated transcription binding protein response elements from the rat somatostatin gene (Vallejo, et al., 1992, J. Biol. Chem. 267(18):12868-12875) and human chorionic gonadotropin (hCG) gene (Deutsch, et al., 1987, J. Biol. Chem. 262(25):12169-12174), was prepared following standard cloning procedures (Sambrook et al., supra). In brief, the plasmid was constructed by inserting a double stranded oligonucleotide linker, CRE-2 (SEQ ID NO:22), containing the rat somatostatin CRE sequence (5′-TTGGCTGACGTCAGAGAGAG-3′; SEQ ID NO:20), the human chorionic gonadotropin (hCG) CRE sequence (5′-AAATTGACGTCATGGTAA-3′; SEQ ID NO:21), and MluI and BglII cohesive ends, into the MluI and BglII sites, respectively, of plasmid pGL2-promoter (Promega, Madison, Wis., catalog no. E1631). The CRE-2 linker had the structure:

CRE-2 Linker

| | |
|---|---|
| | M1uI RAT SOMATOSTATIN hCG Bg1II |
| CREA | 5′-CGCGTTTGGCTGACGTCAGAGAGAGAAAATTGACGTCATGGT |

AAA- 3′

| | |
|---|---|
| CREB | 3′-AAACCGACTGCACTCTCTCTCTTTAACTGCAGTACCATTTCT |

AG-5′

The CRE-2 oligonuceotide linker was prepared by first synthesizing the CREA and CREB oligonucleotides, including the MluI and BglII cohesive ends, using a DNA synthesizer Model 370 from PE-Biosystems (Foster City, Calif.). The CREA and CREB oligonucleotides were then phosporylated at their 5′ ends for cloning purposes using [γ-³²P]-ATP (300 pmoles, specific activity 10 Ci/mmole) and T4-polynucleotide kinase (Gibco-BRL, 2 units). To anneal the oligonucleotides, 100 pmoles each of ³²P-labeled CREA and CREB were mixed in 50 mM Tris-HCl, pH 7.5, 10 mM MgCl₂, the solution was heated at 95° C. for 3 min., and then allowed to cool to ambient temperature over approximately 30 min. to permit annealing of the strands to form the double-stranded CRE-2 linker containing MluI and BglII cohesive ends. The CRE-2 linker was then ligated to the pGL2-promoter plasmid linearized with the restriction enzymes MluI and BglII by standard cloning procedures. The ligation mixture was then used to transform DH5-α competent cells (Gibco-BRL, cat. no. 18258-012) according to the vendor's instructions. The plasmid pGL2-promoter/CRE was then isolated from the transformed E. coli cells, purified by cesium chloride gradient centrifugation (Sambrook, et al., supra), and used as a luciferase reporter plasmid.

Following the protocol provided by Gibco-BRL for the LipofectAMINE PLUS transfection kit (Gibco-BRL, catalog no. 10964-013), human embryonic kidney 293 cells (ATCC CRL 1573) were seeded at 6×10⁵ cells per well in polylysine coated 6-well plates (Becton Dickinson Labware, Franklin Lakes, N.J.). The incubation medium was Dulbecco's Modified Eagles Medium (DMEM)/F12 (3:1) medium (Gibco-BRL, Formula no. 93-0152DK) containing 5% fetal bovine serum (Hyclone, Logan, Utah, catalog no. SH30070.03) and 1× Penicillin-Streptomycin-Fungizon (Gibco-BRL, catalog no. 15240-062). The cells were incubated at 37° C. under 5% CO₂ for 18 hrs. A mixture containing 0.5 μg of the plasimd pcDNA3.1-bMC4R, 0.5 μg of pGL2-promoter/CRE, 6 μl PLUS reagent (Gibco-BRL, catalog no. 11514-015), and 4 μl of LipofectAMINE reagent (Gibco-BRL, catalog no. 10964-013) in serum free DMEM/F12 (3:1) medium (Gibco-BRL, Formula no. 93-0152DK) was added to each well. After 3 hrs incubation at 37° C. under 5% CO₂, the incubation medium was replaced with complete medium DMEM/F12 (3:1) containing 5% fetal bovine serum. The transiently transfected human embryonic kidney 293 cells were then grown for 24 hrs at 37° C. under 5% CO₂ before the cells were treated with 0, 1, and 10 nM of human α-MSH (Peninsula Labs, Inc., Belmont, Calif.; catalog no. 7251). The incubation medium used for α-MSH treatment contained 0.1% Bovine Serum Albumin (Sigma Chemical Co., St. Louis, Mo., catalog no. A-4503) in DMEM/F12 (3:1), and incubation was carried out at 37° C. for 18 hrs under 5% CO₂. At the end of the incubation, the medium was removed and the cells were lysed with 400 μl of lysis buffer (Luciferase Reporter Gene Assay Kit, Boehringer-Mannheim Biochemicals, Indianapolis, Ind., catalog no. 1669893) at room temperature. Thirty-five microliters of lysate were mixed with 80 μl of luciferin solution (Boehringer-Mannheim Luciferase Reporter Gene Assay Kit, supra). The luciferase activity was measured using a luminometer (MLX Microtiter Plate Luminometer, Dynex Technologies, Inc., Chantilly, Va.). The results are shown in Table 3.

TABLE 3
Functional activity of bovine
MC4-R in transiently transfected human
embryonic kidney 293 cells

| α-MSH | Luciferase |
|---|---|
| Concentration | Activity |
| 0 nM | 8.19 |
| | 7.78 |
| 1 nM | 24.80 |
| | 26.67 |
| 10 nM | 35.13 |
| | 37.13 |

As shown in Table 3, the cloned bovine MC4-R expressed in human embryonic kidney 293 cells exhibited functional activity, and was activated by the melanocortin agonist α-MSH, similar to observations on human and rat MC4-R functional activities in 293 cells (data not shown).

The invention being thus described, it is obvious that the same can be varied in many ways. Such variations are not to be regarded as a departure from the spirit and scope of the present invention, and all such modifications as would be obvious to one skilled in the art are intended to be included within the scope of the following claims.

TABLE 1

| | | start |
|---|---|---|
| hMC4R (L08603) | (1) | ----------------------------------------ATGGTGAACT |
| Rat MC4 RNU67863 | (105) | ACGGGGGGGGGGGGGAGGATTCGAATCCAGCTGCTGCAGGAAGATGAACT |
| hMC4R (L08603) | (11) | CCACCCA---CCGTGGGATGCACACTTCTCTGCACCTCTGGAACCGCAGC |
| Rat MC4 RNU67863 | (155) | CCACCCACCACCATGGCATGTATACTTCCCTCCACCTCTGGAACCGCAGC |
| hMC4R (L08603) | (58) | AGTTACAGACTGCACAGCAATGCCAGTGAGTCCCTTGGAAAAGGCTACTC |
| Rat MC4 RNU67863 | (205) | AGCCACGGGCTGCACGGCAATGCCAGCGAGTCTCTGGGGAAGGGGCACTC |
| | | MC4-A |
| hMC4R (L08603) | (108) | TGATGGAGGGTGCTACGAGCAACTTTTTTGTCTCTCCTGAGGTGTTTGTGA |
| Rat MC4 RNU67863 | (255) | AGACGGAGGATGCTATGAGCAACTTTTTGTCTCCCCCGAGGTTTGTGTGA |
| hMC4R (L08603) | (158) | CTCTGGGTGTCATCAGCTTGTTGGAGAATATCTTAGTGATTGTGGCAATA |
| Rat MC4 RNU67863 | (305) | CTCTGGGTGTCATAAGCCTGTTGGAGAACATTCTAGTGATCGTGGCGATA |
| hMC4R (L08603) | (208) | GCCAAGAACAAGAATCTGCATTCACCCATGTACTTTTTCATCTGCAGCTT |
| Rat MC4 RNU67863 | (355) | GCCAAGAACAAGAACCTGCACTCACCCATGTACTTTTTCATCTGTAGTCT |
| hMC4R (L08603) | (258) | GGCTGTGGCTGATATGCTGGTGAGCGTTTCAAATGGATCAGAAACCATTA |
| Rat MC4 RNU67863 | (405) | GGCTGTGGCGGACATGCTGGTGAGCGTTTCGAACGGGTCAGAAACCATCG |
| hMC4R (L08603) | (308) | TCATCACCCTATTAAACAGTACAGATACGGATGCACAGAGTTTCACAGTG |
| Rat MC4 RNU67863 | (455) | TCATCACCCTGCTAAACAGTACGGACACGGACGCCCAGAGCTTCACCGTG |
| hMC4R (L08603) | (358) | AATATTGATAATGTCATTGACTCGGTGATCTGTAGCTCCTTGCTTGCATC |
| Rat MC4 RNU67863 | (505) | AATATTGATAATGTCATTGACTCTGTGATCTGTAGCTCCTTGCTCGCATC |
| hMC4R (L08603) | (408) | CATTTGCAGCCTGCTTTCAATTGCAGTGGACAGGTACTTTACTATCTTCT |
| Rat MC4 RNU67863 | (555) | CATTTGCAGCCTGCTTTCCATTGCAGTGGACAGGTATTTCACTATCTTTT |
| hMC4R (L08603) | (458) | ATGCTCTCCAGTACCATAACATTATGAOAGTTAAGCGGGTTGGGATCATC |
| Rat MC4 RNU67863 | (605) | ACGCGCTCCAGTACCATAACATTATGACGGTTAGGCGGGTCCGGATCATC |
| hMC4R (L08603) | (508) | ATAAGTTGTATCTGGGCAGCTTGCACGGTTTCACGCATTTTGTTCATCAT |
| Rat MC4 RNU67863 | (655) | ATCAGTTGTATCTGGGCAGCTTGCACAGTATCGGGCGTTCTTTTTATCAT |
| hMC4R (L08603) | (558) | TTACTCAGATAGTAGTGCTGTCATCATCTGCCTCATCACCATGTTCTTCA |
| Rat MC4 RNU67863 | (705) | TTACTCGGACAGCAGCGCTGTCATCATCTGCCTCATTACCATGTTCTTCA |
| | | MC4-B |
| hMC4R (L08603) | (608) | CCATGCTGGCTCTCATGGCTTCTCTCTATGTCCACATGTTCCTGATGGCC |
| Rat MC4 RNU67863 | (755) | CCATGCTGGTTCTCATGGCCTCTCTCTATGTCCACATGTTCCTGATGGCG |
| hMC4R (L08603) | (658) | AGGCTTCACATTAAGAGGATTGCTGTCCTCCCCGGCACTGGTGCCATCCG |
| Rat MC4 RNU67863 | (805) | AGGCTTCACATTAAGAGGATCGCTGTCCTCCCGGGCACGGGTACCATCCG |
| hMC4R (L08603) | (708) | CCAAGGTGCCAATATGAAGGGAGCGATTACCTTGACCATCCTGATTGGCG |
| Rat MC4 RNU67863 | (855) | ACAGGGTGCCAACATGAAGGGCGCAATTACCTTGACCATTCTGATTGGAG |
| hMC4R (L08603) | (758) | TCTTTGTTGTCTGCTGGGCCCCATTCTTCCTCCACTTAATATTCTACATC |
| Rat MC4 RNU67863 | (905) | TGTTTGTTGTCTGCTGGGCCCCGTTTTTCCTCCATTTACTGTTCTACATC |
| hMC4R (L08603) | (808) | TCTTGTCCTCAGAATCCATATTGTGTGTGCTTCATGTCTCACTTTAACTT |
| Rat MC4 RNU67863 | (955) | TCTTGTCCTCAGAATCCATACTGCGTGTGCTTCATGTCTCATTTTAACTT |
| hMC4R (L08603) | (858) | GTATCTCATACTGATCATGTGTAATTCAATCATCGATCCTCTGATTTATG |
| Rat MC4 RNU67863 | (1005) | GTATCTCATACTGATCATGTGTAACGCTGTCATCGACCCTCTCATTTATG |
| hMC4R (L08603) | (908) | CACTCCGGAGTCAAGAACTGAGGAAAACCTTCAAAGAGATCATCTGTTGC |
| Rat MC4 RNU67863 | (1055) | CCCTGCGGAGTCAAGAACTGAGGAAAACCTTCAAAGAGATCATCTGTTTC |
| | | stop |
| hMC4R (L08603) | (958) | TATCCCCTGGGAGGCCTTTGTGACTTGTCTAGCAGATATTAA-------- |
| Rat MC4 RNU67863 | (1105) | TACCCCCTGGGAGGCATCTGTGAGTTACCTGGCAGGTATTAAGTGGGGAC |

TABLE 2

| | | |
|---|---|---|
| | | 1 50 |
| Human MC4R | (1) | MVNSTHRGMHTSLHLWNRSSYRLHSNASESLGRGYSDGGCYEQLFVSPEV |
| Rat MC4R | (1) | MNSTHHMGMYTSLHLWNRSSHGLHGNASESLGKGHSDGGCYEQLFVSPEV |
| Porcine MC4R | (1) | MNSTHHHGMHTSLHFWNRSTYGLHSNASEPLGKGYSEGGCYEQLFVSPEV |
| Bovine MC4R | (1) | MNSTQPLGMHTSLHSWNRSAHGMPTNVSESLAKGYSDGGCYEQLFVSPEV |
| | | ECD 1 |
| | | 51 100 |
| Human MC4R | (51) | FVTLGVISLLENILVIVAIAKNKNLHSPNYFFICSLAVADMLVSVSNGSE |
| Rat MC4R | (51) | FVTLGVISLLENILVIVAIAKNKNLHSPNYFFICSLAVADMLVSVSNGSE |
| Porcine MC4R | (51) | FVTLGVISLLENILVIVAIAKNKNLHSPMYFFICSLAVADMLVSVSNGSE |
| Bovine MC4R | (51) | FVTLGVISLLENILVIVAIAKNKNLHSPMYFFICSLAVADMLVSVSNGSE |
| | | TMD1 CD 1 TMD2 |
| | | 101 150 |
| Human MC4R | (101) | TIIITLLNSTDTDAQSFTVNIDNVIDSVICSSLLASICSLLSIAVDRYFT |
| Rat MC4R | (101) | TIVITLLNSTDTDAQSFTVNIDNVIDSVICSSLLASICSLLSIAVDRYFT |
| Porcine MC4R | (101) | TIVITLLNSTDTDAQSFTVNIDNVIDSVICSSLLASICSLLSIAVDRYFT |
| Bovine MC4R (101) | TTIVITLLNSTDTDAQSFTDIDNVIDSVICSSLLASICSLLSIAVDRYFT | |
| | | ECD 2 TMD3 |
| | | 151 200 |
| Human MC4R | (151) | IFYALQYHNIMTVKRVGIIISCIWAACTVSGILFIIYSDSSAVIICLITM |
| Rat MC4R | (151) | IFYALQYHNlMTVRRVGIIISCIWAACTVSGVLFIIYSDSSAVIICLITM |
| Porcine MC4R | (151) | IFYALQYHNIMTVKRVGIIISCIWAVCTVSGVLFIIYSDSSAVIICLITV |
| Bovine MC4R | (151) | IFYALQYHNIMTVKRVAITISAIWAACTVSGVLFIIYSDSSAVIICLITV |
| | | CD 2 TDM4 ECD 3 |
| | | 201 250 |
| Human MC4R | (201) | FFTMLALMASLYVHMFLMARLHIKRIAVLPGTGAIRQGANMKGAITLTIL |
| Rat MC4R | (201) | FFTMLVLMASLYVHMFLMARLHIKRIAVLPGTGTIRQGANMKGAITLTIL |
| Porcine MC4R | (201) | FFTMLVLMASLYVHMFLMARLHIKRIAVLPGTGTIRQGANMKGAITLTIL |
| Bovine MC4R | (201) | FFTMLALMASLYVHMFLMARLHIKRIAVLPGSGTIRQGANMKGAITLTIL |
| | | TMD5 CD 3 TMD6 |
| | | 251 300 |
| Human MC4R | (251) | IGVFVVCWAPFFLHLIFYISCPQNPYCVCPMSHFNLYLILIMCNSIIDPL |
| Rat MC4R | (251) | IGVFVVCWAPFFLHLLFYISCPQNPYCVCFMSHFNLYLILIMCNAVIDPL |
| Porcine MC4R | (251) | IGVFVVCWAPFFLHLIFYISCPQNPYCVCFMSHFNLYLILIMCNSIIDPL |
| Bovine MC4R | (251) | IGVFVVCWAPFFLHILIFYISCPQNPYCVCFMSHFNLYLILIMCNSIIDFL |
| | | ECD 4 TMD7 |
| | | 301 333 |
| Human MC4R | (301) | IYALRSQELRKTFKEIICCYPLGGLCDLSSRY- |
| Rat MC4R | (301) | IYALRSQELRXTFKEIICFYPLGGICELPGRY- |
| Porcine MC4R | (301) | IYALRSQELRKTFKEIICCYPLGGLCDLSSRY- |
| Bovine MC4R | (301) | IYALRSQELRKTFKEIICCSPLGGLCDLSSRY- |
| | | CD 4 |

What is claimed is:

1. An isolated bovine melanocortin 4 receptor protein comprising the amino acid sequence shown in SEQ ID NO:2.

2. An isolated nucleic acid fragment comprising a nucleotide sequence encoding said protein of claim 1.

3. An isolated nucleic acid fragment comprising a nucleotide sequence selected from the group consisting of:
(a) nucleotides 283 to 1281 of SEQ ID NO:1;
(b) a nucleotide sequence encoding the same protein as nucleotides 283 to 1281 of SEQ ID NO:1, but which is degenerate in accordance with the degeneracy of the genetic code; and
(c) a nucleotide sequence complementary to (a) or (b).

4. An isolated nucleic acid fragment that:
(a) hybridizes to the complement of nucleotides 283 to 1281 of SEQ ID NO:1 at a temperature in the range of from about 37° C. to about 42° C., a formamide concentration of about 30%, and 5× SSPE, and washing at about 42° C. in 1×-2× SSPE; and
(b) encodes a molecule that exhibits bovine melanocortin 4 receptor protein activity,
with the proviso that said isolated nucleic acid fragment is of bovine origin and comprises a previously unknown nucleotide sequence.

5. An isolated nucleic acid fragment that:
(a) hybridizes to the complement of nucleotides 283 to 1281 of SEQ ID NO:1 at a temperature in the range of from about 50° C. to about 65° C., a formamide concentration of about 50%, and 5× SSPE, and washing at about 50° C. to about 65° C. in 0.5× SSPE; and
(b) encodes a molecule that exhibits bovine melanocortin 4 receptor protein activity, with the proviso that said isolated nucleic acid fragment is of bovine origin and comprises a previously unknown nucleotide sequence.

6. A vector comprising said isolated nucleic acid fragment of claim 2.

7. The vector of claim 6, wherein said isolated nucleic acid fragment is operably linked to regulatory elements required for expression of said nucleic acid fragment.

8. A host cell containing said vector of claim 6.

9. A host cell containing said vector of claim 7.

10. A method for expressing a bovine melanocortin 4 receptor protein in a host cell, comprising:
(a) transfecting or transforming said vector of claim 9 into a suitable host cell; and
(b) culturing said host cell of step (a) under conditions that permit expression of said bovine melanocortin 4 receptor protein in said host cell.

11. The method of claim 10, further comprising recovering said bovine melanocortin 4 receptor protein.

12. The method of claim 11, wherein said bovine melanocortin 4 receptor protein comprises the amino acid sequence shown in SEQ ID NO:2.

13. The host cell of claim 9, wherein said bovine melanocortin 4 receptor protein is present within the plasma membrane of said host cell.

14. A membrane fraction prepared from said host cell of claim 9, comprising said bovine melanocortin 4 receptor protein.

15. The membrane fraction of claim 14, comprising the plasma membrane of said host cell.

16. A method for identifying a substance that binds to a bovine melanocortin 4 receptor protein, comprising:
(a) providing test cells into which has been introduced an expression cassette that directs the expression of said bovine melanocortin 4 receptor protein in said test cells, wherein said bovine melanocortin 4 receptor protein is expressed and is functional;
(b) contacting said test cells with said substance;
(c) contacting otherwise identical control cells, into which said expression cassette has not been introduced, with said substance;
(d) determining the amount of said substance bound to said test cells and said control cells; and
(e) comparing the amount of said substance bound to said test cells with the amount of said substance bound to said control cells,
wherein an increase in the amount of said substance bound to said test cells compared to the amount of said substance bound to said control cells indicates that said substance binds to said bovine melanocortin 4 receptor protein.

17. The method of claim 16, wherein said bovine melancortin 4 receptor protein comprises the amino acid sequence shown in SEQ ID NO:2.

18. A method for identifying a substance that binds to a bovine melancortin 4 receptor protein, comprising:
(a) providing test cells into which has been introduced an expression cassette that directs the expression of said bovine melancortin 4 receptor protein in said test cells, wherein said bovine melancortin 4 receptor protein is expressed and is functional;
(b) exposing said test cells to said substance;
(c) determining the amount of cyclic AMP produced within said test cells;
(d) determining the amount of cyclic AMP produced in otherwise identical control cells into which said expression cassette has not been introduced; and
(e) comparing the amount of cyclic AMP produced in said test cells of step (c) with the amount of cyclic AMP produced in said cells of step (d), wherein an increase in the amount of cyclic AMP produced in said test cells of step (c) compared to the amount of cyclic AMP produced in said cells of step (d) indicates that said substance binds to said bovine melancortin 4 receptor protein.

19. The method of claim 18, wherein said bovine melanocortin 4 receptor protein comprises the amino acid sequence shown in SEQ ID NO:2.

20. A method for determining whether a substance is a potential agonist or antagonist of a bovine melanocortin 4 receptor protein, comprising:
(a) providing test cells into which has been introduced an expression cassette that directs the expression of said bovine melanocortin 4 receptor protein in said test cells, wherein said bovine melanocortin 4 receptor protein is expressed and is functional;
(b) allowing said test cells to grow for a time sufficient to permit said bovine melanocortin 4 receptor protein to be expressed;
(c) exposing said test cells to a labeled ligand of said bovine melanocortin 4 receptor protein in the presence and in the absence of said substance; and
(d) measuring the binding of said labeled ligand to said bovine melanocortin 4 receptor protein of said test cells,
wherein if the amount of binding of said labeled ligand to said bovine melanocortin 4 receptor protein of said test cells is lower in the presence of said substance than in the absence of said substance, then said substance is a potential agonist or antagonist of said bovine melanocortin 4 receptor protein.
